(19) United States
(12) Patent Application Publication
(10) Pub. No.: US 2023/0235296 A1
(43) Pub. Date: Jul. 27, 2023

(54) ENGINEERED MULTICELLULAR ORGANISMS

(71) Applicants: Trustees of Tufts College, Medford, MA (US); University of Vermont and State Agricultural College, Burlington, VT (US)

(72) Inventors: Michael Levin, Beverly, MA (US); Sam Kriegman, Burlington, VT (US); Douglas Blackiston, Medford, MA (US); Josh Bongard, Burlington, VT (US)

(21) Appl. No.: 18/003,705

(22) PCT Filed: Jan. 12, 2021
(86) PCT No.: PCT/US2021/013105

§ 371 Date: Dec. 29, 2022

Related U.S. Application Data
(60) Provisional application No. 63/045,462, filed on Jun. 29, 2020.

(51) Int. Cl. C12N 5/071; A61K 35/545; G01N 33/50; G16B 5/30; B09C 1/10

(52) U.S. Cl. CPC C12N 5/0697; A61K 35/545; G01N 33/5023; G16B 5/30; B09C 1/10; C12N 2510/00; B09C 2101/00

(57) ABSTRACT

Disclosed are engineered multicellular organisms. Also disclosed are systems and methods for designing, preparing, and utilizing the engineered multicellular organisms.

### CROSS-REFERENCE TO RELATED PATENT APPLICATIONS

The present application claims the benefit of priority under 35 U.S.C. 119(e) to U.S. Provisional Application No. 63/045,462, filed on Jun. 29, 2020, the content of which is incorporated herein by reference in its entirety.

### STATEMENT REGARDING FEDERALLY SPONSORED RESEARCH OR DEVELOPMENT

This invention was made with government support under grant HR0011-18-2-0022 awarded by the Department of Defense and grants 0939511 and 1830870 awarded by the National Science Foundation. The government has certain rights in the invention.

### BACKGROUND

The field of the invention relates to engineered multicellular organisms and systems and methods for designing, preparing, and utilizing engineered multicellular organisms. The engineered multicellular organisms may be configured for movement and other physical and biological activities.

Scalable methods for designing and training machines in silico have been described in the art. (See, e.g., Sims, “Evolving 3D Morphology and Behavior by Competition,” Artificial Life IV Proceedings, ed. By R. Brooks & P. Maes, MIT Press, 1994, pp. 28-39; Munk et al., “Topology and Shape Optimization methods using evolutionary algorithms: a review,” Struct. Multidisc. Optim. (2015) 52:613-631; and Cheney et al., “Scalable co-optimization of morphology and control in embodied machines,” J. R. Soc. Interface 15: 20170937). In addition, automatic design of robotic lifeforms using evolutionary algorithms have been described. (See Bongard et al., “Resilient Machines Through Continuous Self-Modeling,” 17 Nov. 2006, Vol. 314, Science; and Lipson et al., “Automatic design and manufacture of robotic lifeforms,” Nature, Vol. 406, 31 Aug. 2000, p. 974-978). However, these same methods of automatic design have not been applied to organic life forms, which may be self-renewing and biocompatible.

Inorganic robotic lifeforms degrade over time and can produce harmful ecological and health side effects. In addition, inorganic robots are relatively large, cannot repair themselves and do not have built-in physiological robustness and other capabilities of living organisms. As such, organic robotic lifeforms are desirable.

Here, we present a method that designs completely biological machines from the ground up. In our disclosed methods, computers automatically design new organic lifeforms in simulation, and the best designed lifeforms are then selected and built by combining together different biological tissues. The methods disclosed herein may be utilized to design a variety of organic lifeforms to safely deliver drugs inside the human body, help with environmental remediation, search for, accumulate, or report on the presence of various chemicals in waterways, or further broaden our understanding of the diverse forms and functions life may adopt to advance exobiology and regenerative medicine.

### SUMMARY

Disclosed are engineered multicellular organisms. Also disclosed are systems and methods for designing, preparing, and utilizing the engineered multicellular organisms.

The engineered multicellular organisms typically comprise an aggregate of cells from any species that comprises, consists essentially of, or consists of passive cells and contractile cells. Preferably, the engineered multicellular organisms move when the contractile cells of the organism are actuated. The engineered multicellular organism may be configured for various applications, which include but are not limited to delivering a therapeutic agent to a subject in need thereof, removing a target substrate from an environment, building other smaller structures either in vitro or in vivo by accreting or debriding them, and/or detecting a target ligand in a sample.

Also disclosed are systems and methods for designing engineered multicellular organisms. The systems comprise and the methods may utilize at least one hardware processor that is programmed to: (i) generate a design space comprising a plurality of configurations of a polycube, the polycube comprising passive voxels, contractile voxels, and null voxels; (ii) simulate movement or the lack thereof of the configurations based on actuation of the contractile voxels of the configurations; and (iii) generate a report reporting movement or lack thereof for the configurations. The hardware processor may be further programmed: (iv) to receive input data corresponding to an input configuration and to evolve the input configuration by randomly modifying the input data to generate modified data corresponding to an evolved configuration and simulate movement or the lack thereof of the evolved configuration based on actuation of the contractile voxels of the evolved configuration; and (v) to select the evolved configuration if the evolved configuration exhibits enhanced movement relative to the input configuration, and optionally, to further evolve the evolved configuration. Alternatively, the hardware processor may be further programmed: (iv) to act up on an existing set of configurations by randomly selecting, copying and modifying any subset from this set and simulating the movement or lack thereof of the new configurations; and (v) to preferentially delete those with lower average movement within the current configuration set, and optionally, to further evolve the current set of configurations.

### BRIEF DESCRIPTION OF THE FIGURES

FIG. 1. Designing and manufacturing reconfigurable organisms. A behavioral goal (e.g., maximize displacement), along with structural building blocks [here, contractile (red) and passive (cyan) voxels], are supplied to an evolutionary algorithm. The algorithm evolves an initially random population and returns the best design that was found. The algorithm is rerun 99 times starting with different random populations, generating a diversity of performant designs in silico (A; See Example, section 5). Performant designs are then filtered by their robustness to random phase modulation of their contractile cells (B; See Example, section 7), constructed in vivo using developing Xenopus cardiomyocyte and epidermal cell progenitors (C-F; See Example, section 8), and placed on the surface of a Petri dish where their behavior is observed and compared to the design's predicted behavior (See Example, section 9). Discrepancies between in silico and in vivo behavior are returned to the evolutionary algorithm in the form of constraints on the kinds of designs that can evolve during subsequent design-manufacture cycles (G; See Example, section 6). Concurrently, tissue layering and shaping techniques are modified such that realized living systems behave more like their virtual model (See Example, section 8).

FIG. 2. Designing reconfigurable organisms. For a given goal, 100 independent evolutionary trials were conducted in silico (A-C). Each colored line represents the velocity of the fastest-moving design within its clade. Each genome (D) dictates anatomy and behavior by determining where and how voxels are combined, and whether they are passive (cyan) or contractile (red; E). Genomes simulate a developmental process and are described in more detail in See Example, section 4. The differing behavioral traces produced by a design (F) are a result of randomly perturbing the actuation of each contractile cell during each evaluation period. The behavioral traces all originate from the same position (blue) but diverge over time until their final destination (red). (G) During one evaluation period, after settling under gravity for 1 s, compressed and expanded contractile voxels are shown in red and green, respectively. Because the genotype is scale-free, the anatomical resolution of any design can be increased (H) while preserving geometry (but not necessarily behavior). When all evolutionary trials complete, the most performant design from each trial is extracted (I). The robust design passed to the next stage of the pipeline moves, on average, more rapidly (red curve) than the average speed of the other 99 designs (gray curve).

FIG. 3. Manufacturing reconfigurable organisms. (A) Aggregation of pluripotent blastula cells harvested from X. laevis embryos. (B) Shaping results in 3D representations of the evolved in silico designs. (C) Layering of cardiac progenitor cells results in contractile cardiomyocyte tissue at specific locations, visualized by red fluorescent lineage tracer. (D) Time-lapse imaging of self-locomotion in an aqueous environment. (E) Emergent behavior of debris aggregation by an individual within the environment and (F) by groups of reconfigurable organisms over a 24-h period. (See Example, section 10.4). (Scale bars: 500 μm for A-E and 5 mm for F, respectively.)

FIG. 4. Transferal from silico to vivo. The first design selected for fabrication and specific hypothesis testing (A) was the most robust yet stable and energy efficient configuration of passive (epidermis; green) and contractile (cardiac; red) tissues found by the evolutionary algorithm. The design was evaluated 25 times for 1 min of simulation time, resulting in 25 movement trajectories (pink curves in C). Six reconfigurable organisms were built which embodied this design (e.g., B. (See Example, section 9). Three were evaluated four times and the other three were evaluated five times for 10 min each (27 blue curves in C). The organisms' direction of movement matched the design's predicted direction of movement (P<0.01; details in See Example, section 9). To determine whether the organisms' movement was a result of chance or due to the design's evolved geometry and tissue placement, geometry and tissue distribution was altered by rotating the design 180° about its transverse plane (D) and evaluating it another 25 times in silico (pink curves in F). Each of the six organisms were likewise inverted (E): four were evaluated five times while the remaining two were only evaluated once (22 blue curves in F). Inverting the design significantly reduces its net displacement (P<0.001), as did inverting the organisms (P<0.0001).

FIG. 5. Pipeline diagram. The pipeline goes from left to right—from input goal to in-silico design to in-vivo output, and back. The pipeline continues to loop indefinitely, or until some external termination condition is reached.

FIG. 6. The fastest designs from the first pass of the pipeline for locomotion. During the first pass of the pipeline for the goal behavior of locomotion, designs were evaluated in an Earth-like terrestrial environment and evolution was permitted to finely tune the phase-modulation (from a global signal) of actuation in each voxel. Under these conditions of precise actuation, and despite an objective explicitly minimizing the number of contractile voxels (red), the best designs (defined by their locomotive ability) did not utilize any passive material. There was also a strong convergence in shape: many independent runs converged to similar geometries. The rainbow streak below each design is its behavior—from initial (blue) to final position (red)—, superimposed, shifted and scaled to fit on the image (each streak is identically scaled). The run champions for runs 1 through 100 (with random seeds 1-100) are pictured in row-major order.

FIG. 7. The fastest designs from the second pass of the pipeline for locomotion. The second pass of the pipeline for locomotion incorporated a few additional constraints: First-order hydrodynamics were added, tissue density was decreased (made lighter), tissue elasticity was decreased (made softer), and actuation was randomized (each voxel has a randomly-assigned phase-offset which is fixed in all descendants). Because actuation is provided by fewer and less-coordinated active cells, the behavioral trajectories (rainbow streaks below the design) have smaller amplitude, and the final displacement was decreased compared to the first pass (FIG. 6). Although designs were allowed to settle under gravity for one second before the evaluation period (and actuation) begins, an appreciable portion of the evaluated behavior sometimes began with the design settling to the floor (falling blue portions of the rainbow curves). (Behavioral streaks are scaled identically within this figure, but different from those in FIG. 6.) These additional constraints increased geometric diversity, promoted the use of passive (cyan) tissue, and generated behavior that better matched that of the biological representations.

FIG. 8. The design filters and selection criteria. The most performant design from each evolutionary run (the 100 designs in FIG. 7) are filtered in order to select the most promising one (in terms of estimated transferability, buildability, and scalability) for manufacture. The most performant designs are first ranked by their median performance (velocity) under random actuation (robustness filter; A). Then, in rank order, each design is evaluated by the three conditionals (B1-B3) in the build filter (B), which removes designs that are not suitable for the current build method, and/or those which are not scalable to more complex tasks in future deployments.

FIG. 9. The most robust design to pass through the build filter: The champion of run 52 (locomotion; second pass) is drawn floating (A, B), and resting on the surface in simulation (C).

FIG. 10. Manufacturing cardiomyocyte-driven reconfigurable organisms. mRNA constructs are delivered to cleave stage Xenopus embryos which inhibit multi-ciliated epithelial cell differentiation and enable tracking of heart muscle tissue, allowing multiple cell types to be combined and intelligently shaped (A). Tissues are layered sequentially, first with an underlying layer of unspecified epithelium upon which cardiac progenitor tissue is deposited (B). A second layer of unspecified epithelium is layered and allowed to heal for an hour (C). Shaping is then applied to contractile regions using a microcautery electrode to produce the final shape (D).

FIG. 11. Determining minimal concavity when shaping reconfigurable organisms. To optimize the build filter when selecting in silico designs for biological sculpting, spherical biobots were subjected to progressively smaller shaping gaps to evaluate the minimal size which persists across the organisms lifespan. Gaps of 100 microns or larger (white line) represented the smallest concavity which was stable at the time of shaping (A, Ai) and across at least five successive days of development (B, Bi).

FIG. 12. Quantifying the structural match. Lineage labeled organisms were imaged in multiple orientations (A) using a FITC filter set to visualize epidermal tissue (green) and a TRITC filter set to visualize cardiac progenitor (red) tissue. K-means clustering was used to classify each pixel as one of three tissue types: contractile, passive, or none (B). The Hausdorff distance between in vivo and in silico pixels (C) was calculated for each tissue type separately, in terms of euclidean distance in microns. Overall structural error is taken to be the larger of the two Hausdorff distances, computed for the two tissue types. The design is automatically cropped and rotated in 3D space to find the 2D perspective with the smallest error.

FIG. 13. Automatic self-repair in reconfigurable organisms. One week-old non-ciliated reconfigurable organisms were subjected to mechanical laceration with microsurgery forceps (A, B). Each individual was filmed with time lapse imaging, every five seconds, for 10 minutes. In all cases, the biological representations were able to close the wound and survive through the remainder of the experiment (A′, B′). Red arrows indicate the site of mechanical damage. Organisms were able to heal much larger wounds during repair experiments than during their initial shaping (FIG. 11). This is because shaping requires subtraction of tissue over a longer period (which physically inhibits the wound from shutting) while repair involves a single laceration delivered in a short duration. Also, in repair experiments, the organisms possess a fully developed epidermis (which is expected to close wounds) compared to shaping experiments where all cells are embryonically derived and are still in the process of differentiation.

FIG. 14. Spontaneous collective behavior and particle aggregation in silico. Collective behavior of multiple designs interacting with yellow particulate matter in the form of 2×2×1 voxel particles (A). Five designs (locomotion run champions, from the second pass; FIG. 7) are initially placed amid a 15×15 lattice of the debris (B). One of the designs (run 16) travels relatively straight, while the other four move in elliptical orbits, often colliding and coupling for multiple revolutions along a new orbit before collision with a third causes them to detach along a transposed version of their soloist trajectory (C). There was no top-down selection for such connections, they occur spontaneously and affect the manner in which the duo interacts with the external objects. In terms of clearing debris, the performance of the interlocked pair is sometimes more efficient than the sum of its parts.

FIG. 15. Aggregation of carmine particles occurs only in the presence of organisms. In the absence of reconfigurable organisms, carmine dye does not self aggregate after 1 or 24 hours (A, B). However, in the presence of self locomoting organisms, movement of dye particles by individuals can be observed after one hour (C), and collective aggregation is observed by 24 h (D). Scale bar indicates 1 mm.

FIG. 16. Explicit selection for object manipulation in silico. A new goal was input into the pipeline: maximize displacement of a 2×2×2 voxel object (yellow), during an evaluation period of 30 sec. Sixteen independent evolutionary runs (with random seeds 1-16) produced sixteen run champs (A). In some designs, a primitive end-effector—a notch in the corner of the body—evolved to hold and manipulate the object (B, C).

FIG. 17. From spontaneous to explicitly-optimized object transportation in silico. Some designs evolved for displacement reduced hydrodynamic drag via a hole through the center of their transverse plane (e.g., the champion of run 15; A). This more complex topology was realized in vivo (B) but was not layered with contractile tissue. In simulation, this emergent feature was exapted as a pouch to store and transport objects (the 2×2×2 yellow “pills” in C). This realization led to a reformulation of the goal behavior for a subsequent round of evolution, in which pouches—with a free-floating pill inside them—were explicitly incorporated as a design constraint (cross-sectional view in D), and the new goal of maximizing the Euclidean distance of the carried object was employed. This yielded evolved object transport in silico (E) and (F). Sixteen runs were performed with this new constraint and objective. The run 8 champion (seed=8) at the end of 350 generations is shown in D and E.

FIG. 18. Object expulsion in silico. A non-locomotion-based goal was supplied to the pipeline: thrown object distance (48). Using an additional objective that maximizes surface-area to volume ratio, limbs evolved which were used to throw a pink 2×2×2 object. For visibility, the voxels in this figure are colored by their angle relative to the ground plane, where green denotes parallel (angle zero), cooler colors (cyan and blue) denote voxel rotations into the right hand side (more or less) of the frame up to −π, and warmer colors (yellow, orange and red) denote voxel rotations into the left hand side up to +π. The run 2 champion (seed=2) at the end of 1000 generations is pictured. However, this design relies on finely-tuned actuation (which is unlikely to transfer) and fails to pass through the build filter because it is 100% muscle.

FIG. 19. The encoding bias. Explicit geometry optimization (passive tissue only) within a 10-by-10-by-3 workspace. We chose seven unique target shapes—the voxelyzed letters “X”, “E”, “N”, “O”, “B”, “T”, and “S”—and employed the same indirect encoding (CPPNs; (37)), optimization algorithm (AFPO; (19)), and hyperparameters as in the main paper. Each shape can be represented as a bitstring with length 300, where each element corresponds to the presence (1) or absence (0) of a voxel at one of the 300 cartesian coordinates in the workspace. The objective function measures the Hamming distance between a given shape and the target. We re-optimized with a 10 times larger population size (500) and parallelized across 10 CPUs, which produced much more accurate solutions in the same wall-clock time (about 25 minutes per target). This suggests that shape (if not behavioral) complexity scales with population size. The difficulty of realizing certain target shapes with this encoding, is due to the particular activation functions we used— sin( ), abs( ), square( ), sqrt(abs( ))—which tend to produce patterns that gradually vary in space, and thus bias search away from abrupt perimeter changes, such as the square cutaways and holes in “E”, “B”, and “S”. This in-silico bias toward round edges mimics an in-vivo bias in our experiments: the pooled stem cells form a sphere during incubation, and naturally round-off abruptly carved edges after shaping. In future design-manufacture cycles, if particular structures are known to be obtainable in vivo and useful for a given task environment (e.g., long appendages for reaching), mathematical functions that promote such features (e.g., a step function) can be included in the in-silico design.

FIG. 20. Stochastic gradient descent directly applied to the design problem. A policy gradient method (44) was paired with a popular SGD optimizer (45) and directly applied to the design problem given a random actuation pattern (seed=1). The solution returned by this algorithm is shown from four different perspectives, with a yellow star drawn above one of the voxels for reference. It is less performant (4.6 body lengths/min) than the solutions found by the evolutionary algorithm. But more importantly, it is not manufacturable using the current build method: it fails all three conditionals of the build filter (FIG. 8).

FIG. 21. Algorithm analysis. The optimal design was identified in small solution spaces for five different random actuation patterns (seeds 1-5). The optimal designs in each workspace (A)(2×2×2), (B)(3×2×2), and (C)(3×3×2) are pictured under their corresponding random seed. The evolutionary algorithm used in this paper (green curves; EA) is plotted alongside a policy gradient method (44) which used stochastic gradient descent (blue curves; SGD). Because SGD tends to prematurely converge to a suboptimal design, the algorithm was then modified to restart search from a new random initialization, every 1000 evaluations (orange curves; SGDre). The mean performance of each algorithm (relative to that of the optimal solution) is shown as a solid line. The shaded areas around the lines are bootstrapped 68% confidence intervals of the mean (which for normally distributed random variables, corresponds to plus/minus one standard deviation). The table at the bottom lists the total number of configurations in each workspace, not accounting for isomorphisms, such as translations or certain rotations, or those which result from taking the largest connected component to be the design.

FIG. 22. Scaling the pipeline. Designs were “carved” in silico from 8-by-8-by-7 workspace (448 voxel “cells”) but the organisms were carved in vivo from a 10000 cell sphere, and their final geometries contain about 5000 cells. However, because the genetic encoding is scale-free, evolved designs (A) can be scaled to a higher resolution in silico (B-F) while preserving geometry, but not necessarily behavior. To determine the behavior of anatomically-upscaled designs, additional simulations are required, the CPU-time of which increases with each virtual voxel. Thus upscaling phenotypes can significantly slow the pace of evolution. However, a GPU-accelerated version of the simulator was recently released which, by updating voxels in parallel, may alleviate this concern.

### DETAILED DESCRIPTION

The following discussion is presented to enable a person skilled in the art to make and use embodiments of the disclosure. Various modifications to the illustrated embodiments will be readily apparent to those skilled in the art, and the generic principles herein can be applied to other embodiments and applications without departing from embodiments of the disclosure. Thus, embodiments of the disclosure are not intended to be limited to embodiments shown, but are to be accorded the widest scope consistent with the principles and features disclosed herein. The following detailed description is to be read with reference to the figures. The figures, which are not necessarily to scale, depict selected embodiments and are not intended to limit the scope of embodiments of the disclosure. Skilled artisans will recognize the examples provided herein have many useful alternatives and fall within the scope of embodiments of the disclosure.

### Definitions and Terminology

Disclosed are engineered multicellular organisms and systems and methods designing, preparing, and utilizing the engineered multicellular organisms. The disclosed subject matter may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “a cell” should be interpreted to mean “one or more cells.” As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

Engineered Multicellular Organisms

Disclosed are engineered multicellular organisms. Also disclosed are systems and methods for designing, preparing, and utilizing the engineered multicellular organisms.

The engineered multicellular organisms typically comprise an aggregate of cells. The aggregate of cells may comprise one or more different cell types. In some embodiments, the aggregate of cells comprises, consists essentially of, or consists of passive cells and contractile cells. Suitable passive cells may include, but are not limited to, epithelial cells or progenitor cells thereof. Suitable contractile cells may include, but are not limited to, cardiac cells or progenitor cells thereof.

Optionally, the engineered multicellular organisms may meet at least one of the following criteria: (i) the organism comprises less than about 1000 total cells, or less than about 900, 700, 600, 500, 400, 300, 200, or 100 cells (or the organism comprises a number of cells within a range bounded by any of these values (e.g., 100-1000 cells); and (ii) the organism has an effective diameter of less than about 2 mm, or less than about 1.5 mm, 1.0 mm, 0.9 mm, 0.8 mm, 0.7 mm, 0.6 mm, 0.5 mm, 0.4 mm, 0.3 mm, 0.2 mm, or 0.1 mm (or the organism has an effective diameter within a size range bounded by any of these values (e.g., 0.1-1.0 mm).

The engineered multicellular organisms preferably are self-motile and move when the contractile cells of the organisms are actuated (e.g., when the contractile cells contract and relax). In some embodiments, the contractile cells of the organisms may be actuated by electrical stimulation or optogenetics where the contractile cells have been genetically modified to express light-sensitive ion channels.

In some embodiments, the engineered multicellular organisms are in contact with a surface and move, for example linearly, when the contractile cells of the organisms are actuated. Preferably, the organisms move linearly at a rate of at least about 0.1 mm/minute, 0.2 mm/minute, 0.3 mm/minute, 0.4 mm/minute, 0.5 mm/minute, 0.6 mm/minute, 0.7 mm/minute, 0.8 mm/minute, 0.9 mm/minute, 1.0 mm/minute, 1.5 mm/minute, 2.0 mm/minute, 2.5 mm/minute, or 3.0 mm/minute when the contractile cells of the organisms are actuated. In some embodiments, the organisms are self-motile and move only when the organism is in an upright position and not when the organism is in an inverted position.

The aggregate of cells of the engineered multicellular organism may be obtained from pluripotent blastula cells. In some embodiments, the passive cells of the aggregate of cells are obtained from pluripotent blastula cells. In some embodiments, the contractile cells of the aggregate of cells are obtained from pluripotent blastula cells.

Preferably, the engineered multicellular organism have a life-span when placed in an physiologically suitable environment of at least about 3, 4, 5, 6, 7, 8, 9, 10, 11, 12, 13, 14, 15, 16, 17, 18, 19, 20, 21, 22, 23, 24, 25, 26, 27, 28, 29, or 30 days.

The engineered multicellular organisms comprise an aggregate of cells which may be referred to as a plurality of living cells that are cohered to one another. The aggregate of cells forms a three-dimensional shape.

The aggregate of cells of the disclosed organism may comprise one or more different cell types. The cell types of the aggregate may vary depending on the desired shape of the aggregate and/or function of the aggregate. Suitable cell types may include, but are not limited to, passive cells and contractile cells. Suitable passive cells may include, but are not limited to epithelial cells, such as epithelial cells that form linings in cavities and vessels or channels, exocrine and endocrine secretory epithelial cells, epithelial absorptive cells, keratinizing epithelial cells, and extracellular matrix secretion cells. Suitable contractile cells may include, but are not limited to myocytes (e.g., cardiac myocytes), vascular smooth muscle cells, vascular endothelial cells, skeletal muscle, myofibroblasts, and airway smooth muscle cells. Suitable cells also include cells that will differentiate into contractile cells such as stem cells (e.g., embryonic stem cells and adult stem cells), and progenitor cells.

Suitable cells may comprise animal cells and plant cells. Suitable animal cells may include cells from vertebrates (e.g., fish) or invertebrates (e.g., hydra). Suitable cells may include cells from amphibians, such as cells from Xenopus laevis. Suitable cells may include avian cells. Suitable cells also may include mammalian cells, for example, mouse cells or human cells.

The aggregate of cells may comprise, consist essentially of, or consist of passive cells (e.g., endothelial cells) and contractile cells (e.g., myocytes). In some embodiments, the aggregate of cells may comprise or may not comprise additional cell types, such as fibroblasts, neural cells, connective tissue cells (e.g., cell types which make up bone and cartilage), lymphoid cells, parenchymal cells, and hepatocytes.

Suitable cells for the disclosed organisms and methods may include, but are not limited to, stem cells. The term “stem cell” as used herein and as would be understood in the art, refers to an undifferentiated cell which is capable of proliferation and giving rise to more progenitor cells having the ability to generate mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “progenitor cell” as used herein and as would be understood in the art refers to cells that have a cellular phenotype that is more primitive relative to a cell which it can give rise to by differentiation. Progenitor cells may give rise to multiple distinct differentiated cell types or to a single differentiated cell type, depending on the developmental pathway and on the environment in which the cells develop and differentiate. Depending on context, the term “progenitor cell” may be used synonymously with the term “stem cell.”

The term “embryonic stem cell” refers to the pluripotent stem cells of the inner cell mass of the embryonic blastocyst. Such cells can similarly be obtained from the inner cell mass of blastocysts derived from somatic cell nuclear transfer. The term “adult stem cell” or “ASC” refers to any multipotent stem cell derived from non-embryonic tissue, including fetal, juvenile, and adult tissue.

In some embodiments, the engineered multicellular organisms are non-innervated and/or or non-cartilaginous. The multicellular organisms may be described as “engineered” because they are different from naturally occurring organism that arise without the guidance of human ingenuity and modifications. In other words, the multicellular organisms are synthetic and non-naturally occurring, albeit the multicellular organism may utilize endogenous cell:cell signaling and morphogenesis.

The aggregate of cells of the engineered multicellular organisms may comprise cells that have been engineered to express a heterologous molecule. In some embodiments, the passive cells are engineered to express a heterologous protein or secrete specific desired molecules. In some embodiments, the contractile cells are engineered to express a heterologous molecule.

In embodiments in which the aggregate of cells comprises cells that have been engineered to express a heterologous molecule, suitable heterologous molecules that are expressed may include therapeutic agents. Other suitable heterologous molecules may include enzymes that metabolize a target substrate, which may include toxins. Other suitable heterologous molecules may include receptors for a target ligand (e.g., a target ligand sensed by the organism), or sensors of light, heat, and other physical properties in the environment.

Preferably, the engineered multicellular organisms are self-repairing. In some embodiments, if the aggregate of cells is subjected to deaggregation (e.g., physical damage that disrupts aggregation of the cells), the cells will reaggregate to re-form the aggregate of cells.

The engineered multicellular organisms may be configured in order to perform tasks. In some embodiments, the organism is configured for moving a target object (e.g., by pushing a target object). In further embodiments, the organism is configured for moving target objects (e.g., by pushing target objects) and collecting the moved target objections (i.e., aggregating the target objects).

The engineered multicellular organisms may be configured to have a cavity. In some embodiments, the engineered multicellular organisms are configured to have a cavity for capturing and/or transporting a target object.

The engineered multicellular organism may be utilized in a number of applications. In some embodiments, the organisms are utilized in methods for delivering a therapeutic agent to a subject in need thereof, where the method comprises engineering the organisms o to express the therapeutic agent and administering the organism to the subject.

In other embodiments, the engineered multicellular organisms are utilized in methods for removing a target substrate from an environment (e.g., a toxin from an environment). The methods may comprise engineering the organisms to express an enzyme that metabolizes the target substrate and placing the organism in the environment to remove the target substrate from the environment.

In other embodiments, the engineered multicellular organism are utilized in methods for detecting a target ligand in a sample. The methods may comprise engineering the organisms to express a receptor for the target ligand and place the organism in the sample, where the organism generates a signal after the receptor binds the target ligand.

Also disclosed are systems and methods of using the disclosed systems for designing and/or preparing engineered multicellular organisms. The disclosed systems may comprise at least one hardware processor that is programmed to: (i) generate a design space comprising a plurality of configurations of a polycube, the polycube comprising passive voxels, contractile voxels, and null voxels; (ii) simulate movement or the lack thereof of the configurations based on actuation of the contractile voxels of the configurations; and (iii) generate a report reporting movement or lack thereof for the configurations.

In the disclosed systems, movement may be simulated utilizing a physics engine. Preferably, the physics engine: (a) models interactions between adjacent voxels and interactions between voxels and a ground surface plane; (b) simulates volumetric actuation resulting from contractile cells; and (c) detects and resolves collisions between voxels.

In the disclosed systems, movement may be simulated by providing an input, manipulating the input, and providing an output. In some embodiments, movement is simulated by (a) generating an input for the simulation by encoding each configuration as a network that maps the spatial coordinates of a 3D cartesian lattice of the configuration to a single value indicating whether there is a voxel associated with that value (i.e., indicating whether the location is not a null voxel) and whether there is a contractile voxel or passive voxel associated with that value, wherein the input comprises each voxel's coordinate values, and each voxel's coordinate values comprise each voxel's cartesian coordinates and each voxel's radial distance from the center of the 3D cartesian lattice; (b) generating an output for the simulation by applying a function to each voxel's coordinate values based on volumetric actuation resulting from contractile cells.

The disclosed systems may be configured in order to evolve an input configuration and identify an evolved configuration that exhibits better performance. In some embodiments, the hardware processor of the systems is programmed to receive input data corresponding to an input configuration and to evolve the input configuration by randomly modifying the input data of the input configuration to generate modified data corresponding to an evolved configuration and simulate movement or the lack thereof of the evolved configuration based on actuation of the contractile voxels of the evolved configuration. Optionally, the hardware processor further is programmed to select the evolved configuration if the evolved configuration exhibits enhanced movement relative to the input configuration, and optionally, to further evolve the evolved configuration. In other embodiments, the hardware processor of the systems is programmed to act up on an existing set of configurations by randomly selecting, copying and modifying any subset from this set and simulating the movement or lack thereof of the new configurations by translating actuation of the voxels comprising a configuration into forces that move the configuration. Optionally, the hardware processor further is programmed to preferentially delete those with lower average movement within the current configuration set, and optionally, to further evolve the current set of configurations.

The disclosed systems may be configured to apply filters in order to select preferred configuration. In some embodiments, the hardware processor of the systems is programmed to filter configurations through a robustness filter by simulating movement or the lack thereof of the configurations under random actuation and select a configuration based on the robustness of the configuration. In other embodiments, the hardware process further of the systems also is programmed to filter configurations through a build filter by assessing manufacturability of the configurations (i.e., selecting configurations that are most easily manufactured based on the positioning of passive voxels, contractile voxels, and null voxels).

The disclosed technology has several advantages which may include but are not limited to: (1) rational control of multicellular shapes or behaviors/functions; (2) creation of novel forms through automatic design that do not resemble existing organisms; (3) creation of organic robotic lifeforms through cell-based construction using manipulation of two or more progenitor cell types; and (4) creation of functional organic lifeforms that are mobile, capable of herding objects, capable of capturing and transporting (delivering) objects, self-repairing, and potentially having enzymatic function.

The engineered multicellular organisms are novel based on a number of characteristics. For example, the engineered multicellular organisms are autonomous mobile stem cell-based robots that do not require genetic programming or external controllers. The engineered multicellular organisms are able to collect, transport or capture toxins or plastics or organics. Further, the engineered multicellular organisms are potentially self-targeting, self-organizing and self-orienting, and potentially capable of enzymatic digestion.

As such, the engineered multicellular organisms have numerous applications. For example, the engineered multicellular organisms may be utilized in wastewater treatment including industrial, municipal, and environmental settings, such as: (i) environmental cleanup including toxin/plastic/organics removal; (ii) municipal biofilm removal from sewers/tubes; (iii) removal of undesirable microbes or toxins in composted toilets or filtration ponds; (iv) municipal/biopharma—concentration of pharmaceuticals in municipal or biopharma settings; and (v) clean-up in dairy setting to concentrate and convert organics into high-value food ingredients. The engineered multicellular organisms also can be used in water treatment including industrial, agricultural, and biopharma settings, such as: (i) medical or marine applications such as removal biofilm removal from surfaces; (ii) agricultural or municipal application such as non-chlorine removal of E. coli; and (iii) biopharma and bioseparation applications including removal of endotoxins. The engineered multicellular organisms also can be used in medical and dental applications, such as: (i) removal of arterial plaque, clots, dead tissue or targeted delivery of therapeutic agents; (ii) oral rinse to remove dental plaque; and (iii) eyewash to remove floaters. In addition, the engineered multicellular organisms can be used in consumer product applications, such as products to remove make up or dead skin cells or cleaning products to remove slime, mold, and/or mildew.

### Illustrative Embodiments

The following embodiments are illustrative and should not be interpreted to limit the scope of the claimed subject matter.

Embodiment 1. An engineered multicellular organism comprising an aggregate of cells comprising passive cells and contractile cells, wherein the organism moves when the contractile cells are actuated.

Embodiment 2. The organism of embodiment 1, wherein the organism consists of biological material and/or does not comprise any inorganic material, for example as a scaffold.

Embodiment 3. The organism of embodiment 1 or 2, wherein the organism comprises a sensor for detecting a target molecule.

Embodiment 4. The organism of any of the foregoing embodiments, wherein the cells of the organism self-assembly.

Embodiment 5. The organism of any of the foregoing embodiments, wherein the organism moves when the contractile cells are actuated, optionally wherein the organism moves substantially linearly.

Embodiment 6. The organism of any of the foregoing embodiments, wherein the organism moves linearly at a rate of at least about 1 mm/minute when the contractile cells are actuated.

Embodiment 7. The organism of any of the foregoing embodiments, wherein the organism moves only when the organism is in an upright position and not when the organism is in an inverted position.

Embodiment 8. The organism of any of the foregoing embodiments, wherein the passive cells and/or the contractile cells are obtained from pluripotent blastula cells.

Embodiment 9. The organism of any of the foregoing embodiments, wherein the passive cells and/or the contractile cells are engineered to express a heterologous molecule.

Embodiment 10. The organism of embodiment 9, wherein the heterologous molecule is a therapeutic agent.

Embodiment 11. The organism of embodiment 9, wherein the heterologous molecule is an enzyme that metabolizes a target substrate (e.g., a toxin).

Embodiment 12. The organism of embodiment 9, wherein the heterologous molecule is a receptor for a target ligand (e.g., a target ligand sensed by the organism).

Embodiment 13. The organism of any of the foregoing embodiments, wherein the aggregate of cells reaggregates after the aggregate is subjected to deaggregation (i.e., self-repairs).

Embodiment 14. The organism of any of the foregoing embodiments, wherein the organism is configured for moving a target object, optionally wherein the organism comprises a hole for holding the target object.

Embodiment 15. The organism of any of the foregoing embodiments, wherein the organism is configured to have a cavity for capturing and/or transporting a target object.

Embodiment 16. The organism of any of the foregoing embodiments, wherein the organism comprises one or more protrusions that facilitate movement of the organism, optionally vertical protrusions that contact a surface and facilitate movement of the organism.

Embodiment 17. The organism of any of the foregoing embodiments, wherein the organism comprises vertebrate cells.

Embodiment 18. The organism of any of the foregoing embodiments, wherein the organism comprises invertebrate cells.

Embodiment 19. The organism of any of the foregoing embodiments, wherein the organism comprises amphibian cells (e.g., cells from Xenopus laevis).

Embodiment The organism of any of the foregoing embodiments, wherein the organism comprises mammalian cells.

Embodiment 21. The organism of any of the foregoing embodiments, wherein the organism comprises human cells.

Embodiment 22. The organism of any of the foregoing embodiments, wherein the organism comprises mouse cells.

Embodiment 23. A plurality of the organism of any of the foregoing embodiments, wherein the plurality exhibits collective and/or coordinated behavior.

Embodiment 24. A method for delivering a therapeutic agent to a subject in need thereof, the method comprising engineering the organism of any of the foregoing embodiments to express the therapeutic agent and administering the organism to the subject.

Embodiment 25. A method for removing a target substrate from an environment, the method comprising engineering the organism of any of embodiments 1-23 to express an enzyme that metabolizes the target substrate and placing the organism in the environment.

Embodiment 26. A method for detecting a target ligand in a sample, the method comprising engineering the organism of any of embodiments 1-23 to express a receptor for the target ligand and place the organism in the sample, wherein the organism generates a signal after the receptor binds the target ligand.

Embodiment 27. A system for designing the organism of any of the foregoing embodiments, the system comprising at least one hardware processor that is programmed to: (i) generate a design space comprising a plurality of configurations of a polycube, the polycube comprising passive voxels, contractile voxels, and null voxels; (ii) simulate movement or the lack thereof of the configurations based on actuation of the contractile voxels of the configurations; (iii) generate a report reporting movement or lack thereof for the configurations; and optionally (iv) selectively delete configurations that have movement capabilities less than the average movement capabilities of a current set of configurations, selectively copy and randomly modify the remaining configurations, and repeat this process to effect evolution of the set of configurations.

Embodiment 28. The system of embodiment 27, wherein movement is simulated utilizing a physics engine that: (a) models interactions between adjacent voxels and interactions between voxels and a ground surface plane; (b) simulates volumetric actuation resulting from contractile cells; and (c) detects and resolves collisions between voxels.

Embodiment 29. The system of embodiment 27 or 28, wherein movement is simulated by (a) generating an input for the simulation by encoding each configuration as a network that maps the spatial coordinates of a 3D cartesian lattice of the configuration to a single value indicating whether there is a voxel associated with that value (i.e., the location is not a null voxel) and whether there is a contractile voxel or passive voxel associated with that value, wherein the input comprises each voxel's coordinate values, and each voxel's coordinate values comprise each voxel's cartesian coordinates and each voxel's radial distance from the center of the 3D cartesian lattice; (b) generating an output for the simulation by applying a function to each voxel's coordinate values based on volumetric actuation resulting from contractile cells.

Embodiment 30. The system of any of embodiments 27-29, wherein the hardware processor further is programmed to receive input data corresponding to an input configuration and to evolve the input configuration by randomly modifying the input data to generate modified data corresponding to an evolved configuration and simulate movement or the lack thereof of the evolved configuration based on actuation of the contractile voxels of the evolved configuration.

Embodiment 31. The system of any of embodiments 27-29, wherein the hardware processor further is programmed to act up on an existing set of configurations by randomly selecting, copying and modifying any subset from this set and simulating the movement or lack thereof of the new configurations by translating actuation of the voxels comprising a configuration into forces that move the configuration.

Embodiment 32. The system of embodiment 30 or 31, wherein the hardware processor further is programmed to select the evolved configuration if the evolved configuration exhibits enhanced movement relative to the input configuration, and optionally, to further evolve the evolved configuration, and preferentially delete those with lower average movement within the current configuration set, and optionally, to further evolve the current set of configurations.

Embodiment 33. The system of any of embodiments 27-32, wherein the hardware processor further is programmed to filter configurations through a robustness filter by simulating movement or the lack thereof of the configurations under random actuation and select a configuration based on the robustness filter.

Embodiment 34. The system of any of embodiments 27-33, wherein the hardware processor further is programmed to filter configurations through a build filter by assessing manufacturability of the configurations.

Embodiment 35. A method for designing the organism of any of the foregoing embodiments, the method comprising: (i) generating a design space comprising a plurality of configurations of a polycube, the polycube comprising passive voxels, contractile voxels, and null voxels; (ii) simulating movement or the lack thereof of the configurations based on actuation of the contractile voxels of the configurations; (iii) generating a report reporting movement or lack thereof for the configuration; and optionally (iv) selectively delete configurations that have movement capabilities less than the average movement capabilities of a current set of configurations, selectively copy and randomly modify the remaining configurations, and repeat this process to effect evolution of the set of configurations.

Embodiment 36. The method of embodiment 35, wherein movement is simulated utilizing a physics engine that: (a) models interactions between adjacent voxels and interactions between voxels and a ground surface plane; (b) simulates volumetric actuation resulting from contractile cells; and (c) detects and resolves collisions between voxels.

Embodiment 37. The method of embodiment 35 or 36, wherein movement is simulated by (a) generating an input for the simulation by encoding each configuration as a network that maps the spatial coordinates of a 3D cartesian lattice of the configuration to a single value indicating whether there is a voxel associated with that value (i.e., the location is not a null voxel) and whether there is a contractile voxel or passive voxel associated with that value, wherein the input comprises each voxel's coordinate values, and each voxel's coordinate values comprise each voxel's cartesian coordinates and each voxel's radial distance from the center of the 3D cartesian lattice; (b) generating an output for the simulation by applying a function to each voxel's coordinate values based on volumetric actuation resulting from contractile cells.

Embodiment 38. The method of any of embodiments 35-37, further comprising receiving input data corresponding to an input configuration and evolving the input configuration by randomly modifying the input data to generate modified data corresponding to an evolved configuration and simulating movement or the lack thereof of the evolved configuration based on actuation of the contractile voxels of the evolved configuration.

Embodiment 39. The method of any of embodiments 35-37, wherein the hardware processor further is programmed to act up on an existing set of configurations by randomly selecting, copying and modifying any subset from this set and simulating the movement or lack thereof of the new configurations by translating actuation of the voxels comprising a configuration into forces that move the configuration.

Embodiment 40. The method of embodiment 38 or 39, further comprising selecting the evolved configuration if the evolved configuration exhibits enhanced movement relative to the input configuration, and optionally, furthering evolving the evolved configuration, and optionally, to further evolve the evolved configuration, and preferentially delete those with lower average movement within the current configuration set, and optionally, to further evolve the current set of configurations.

Embodiment 41. The method of any of embodiments 35-40, further comprising filtering configurations through a robustness filter by simulating movement or the lack thereof of the configurations under random actuation and selecting a configuration based on the robustness filter.

Embodiment 42. The method of any of embodiments 35-41, further comprising filtering configurations through a build filter by assessing manufacturability of the configurations.

### EXAMPLES

The following examples are illustrative and should not be interpreted to limit the scope of the claimed subject matter.

Title—a Scalable Pipeline for Designing Reconfigurable Organisms

Reference is made to Kriegman, S., Blackiston, D., Levin, M. and Bongard, J., “A scalable pipeline for designing reconfigurable organisms,” Proc. Nat'l. Acad. Sci. USA, Jan. 28, 2020, vol. 117, no. 4, 1853-1859, the content of which is incorporated herein by reference in its entirety.

Abstract

Living systems are more robust, diverse, complex, and supportive of human life than any technology yet created. However, our ability to create novel lifeforms is currently limited to varying existing organisms or bioengineering organoids in vitro. Here we show a scalable pipeline for creating functional novel lifeforms: AI methods automatically design diverse candidate lifeforms in silico to perform some desired function, and transferable designs are then created using a cell-based construction toolkit to realize living systems with the predicted behaviors. Although some steps in this pipeline still require manual intervention, complete automation in future would pave the way to designing and deploying unique, bespoke living systems for a wide range of functions.

### INTRODUCTION

Most modern technologies are constructed from synthetic rather than living materials because the former have proved easier to design, manufacture, and maintain; living systems exhibit robustness of structure and function and thus tend to resist adopting the new behaviors imposed on them. However, if living systems could be continuously and rapidly designed ab initio and deployed to serve novel functions, their innate ability to resist entropy might enable them to far surpass the useful lifetimes of our strongest yet static technologies. As examples of this resistance, embryonic development and regeneration reveal remarkable plasticity, enabling cells or whole organ systems to self-organize adaptive functionality despite drastic deformation (1, 2). Exploiting the computational capacity of cells to function in novel configurations suggests the possibility of creating synthetic morphology that achieves complex novel anatomies via the benefits of both emergence and guided self-assembly (3).

Currently, there are several methods underway to design and build bespoke living systems. Single-cell organisms have been modified by refactored genomes, but such methods are not yet scalable to rational control of multicellular shape or behavior (4). Synthetic organoids can be made by exposing cells to specific culture conditions but very limited control is available over their structure (and thus function) because the outcome is largely emergent and not under the experimenter's control (5). Conversely, bioengineering efforts with 3D scaffolds provide improved control (6-8), but the inability to predict behavioral impacts of arbitrary biological construction has restricted assembly to biological machines that resemble existing organisms, rather than discovering novel forms through automatic design.

Meanwhile, advances in computational search and 3D printing have yielded scalable methods for designing and training machines in silico (9, 10) and then manufacturing physical instances of them (11-13). Most of these approaches employ an evolutionary search method (14) that, unlike learning methods, enables the design of the machine's physical structure along with its behavior. These evolutionary design methods continually generate diverse solutions to a given problem, which proves useful as some designs can be instantiated physically better than others. Moreover, they are agnostic to the kind of artifact being designed and the function it should provide: the same evolutionary algorithm can be reconfigured to design drugs (15), autonomous machines (11, 13), metamaterials (16), or architecture (17).

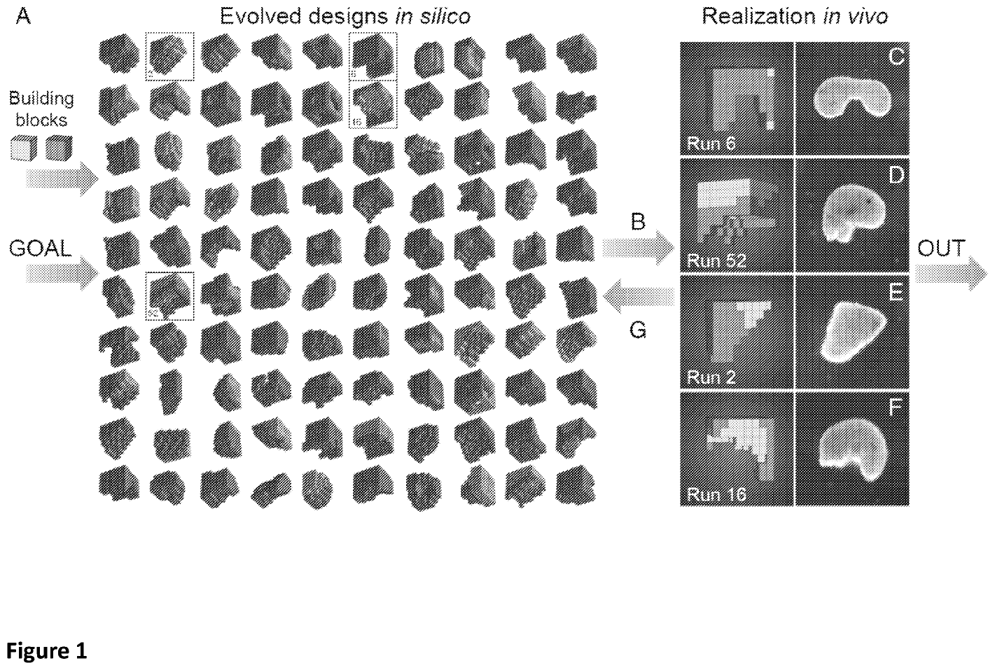

Here, we demonstrate a scalable approach for designing living systems in silico using an evolutionary algorithm, and we show how the evolved designs can be rapidly manufactured using a cell-based construction toolkit. The approach is organized as a linear pipeline that takes as input a description of the biological building blocks to be used and the desired behavior the manufactured system should exhibit (FIG. 1). The pipeline continuously outputs performant living systems that embody that behavior in different ways. The resulting living systems are novel aggregates of cells that yield novel functions: above the cellular level, they bear little resemblance to existing organs or organisms.

Results

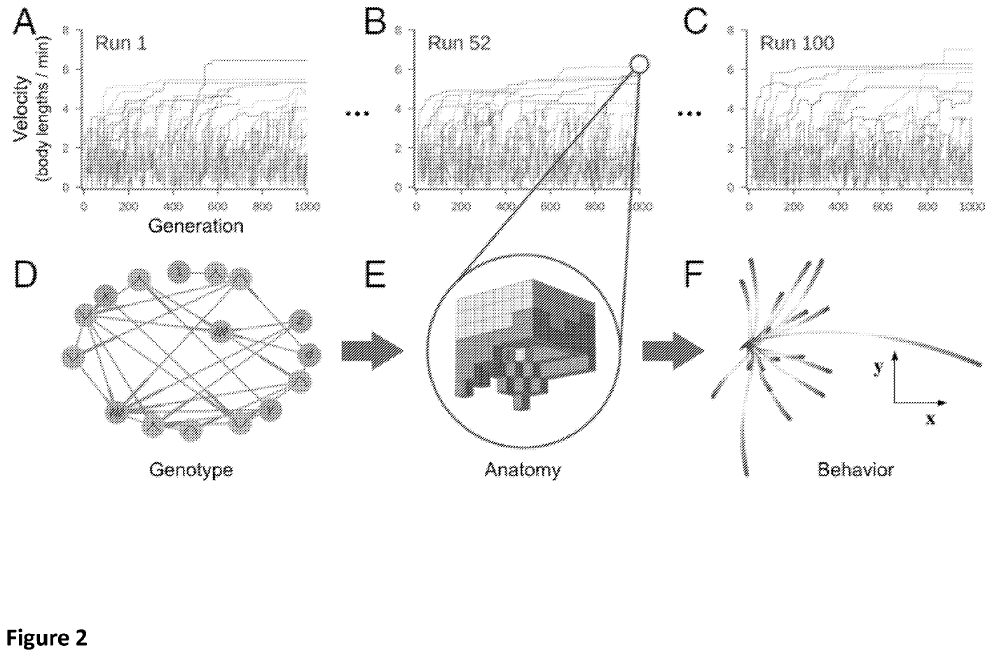

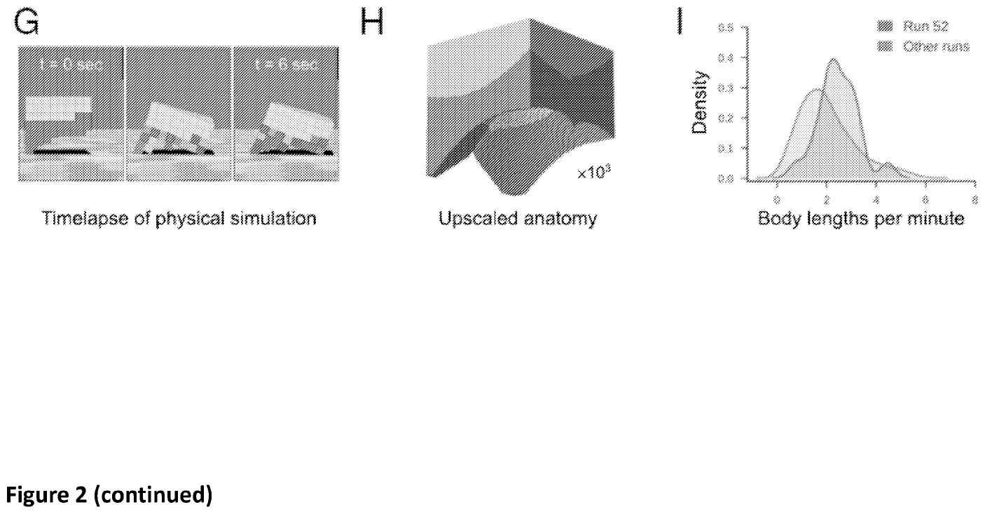

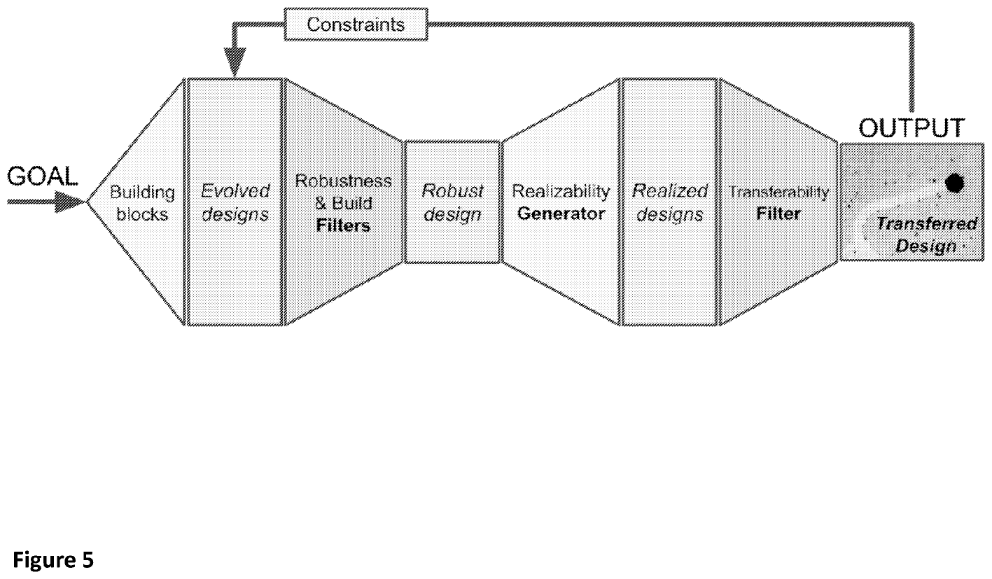

The pipeline is organized as a sequence of generators and filters (See FIG. 5). The first generator is an evolutionary algorithm that discovers different ways of combining the biological building blocks together to realize the desired behavior. A population of random designs are first created. Then, each design is simulated in a physics-based virtual environment and automatically assigned a performance score. Less-performant designs are deleted and overwritten by randomly modified copies of more-performant designs. Repeating this process yields populations of performant and diverse designs (FIG. 2).

The source code necessary for reproducing the computational results reported in this paper can be found at GitHub (github.com/skriegman/reconfigurable_organisms).

As there are likely to be many differences between the simulated and targeted physical environments, performant designs are passed through a robustness filter which only allows passage of designs that sustain the desired behavior in the face of noise. (See Example, section 7). Previous work has shown that noise resistance in simulation is a simple and effective predictor of whether a design will maintain its behavior when instantiated physically (18).

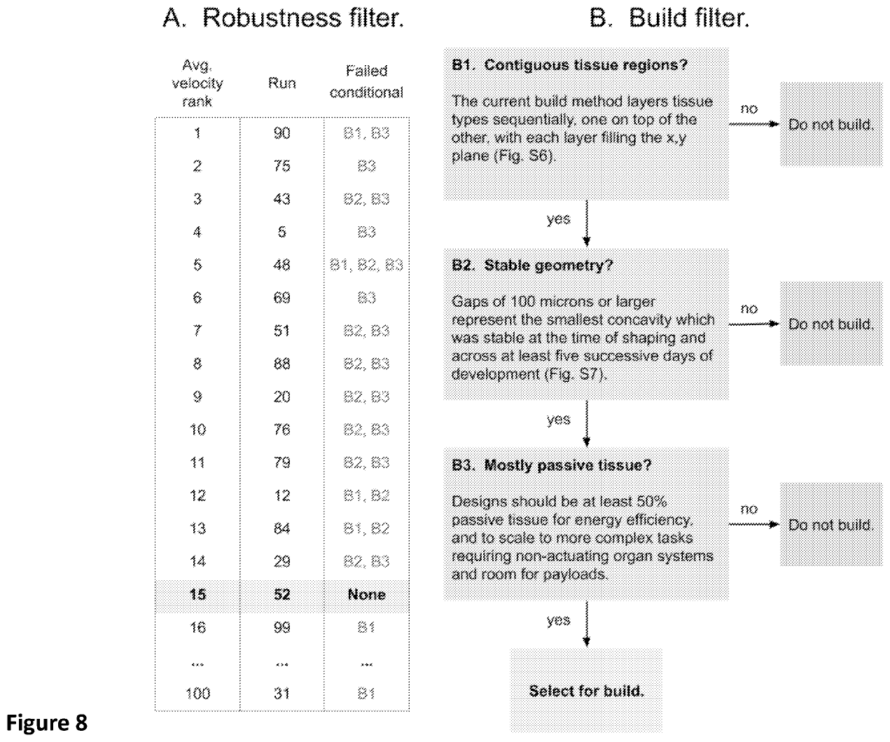

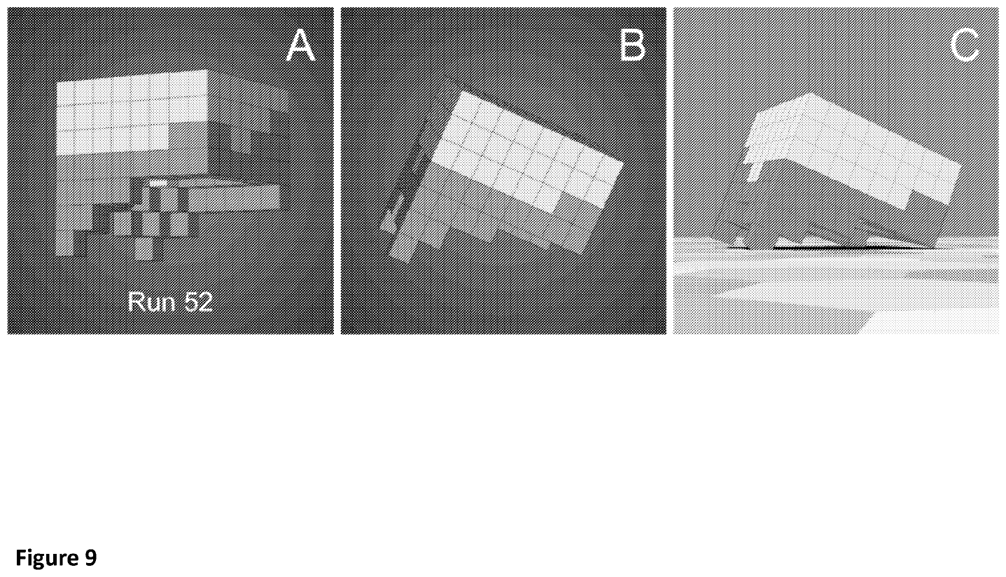

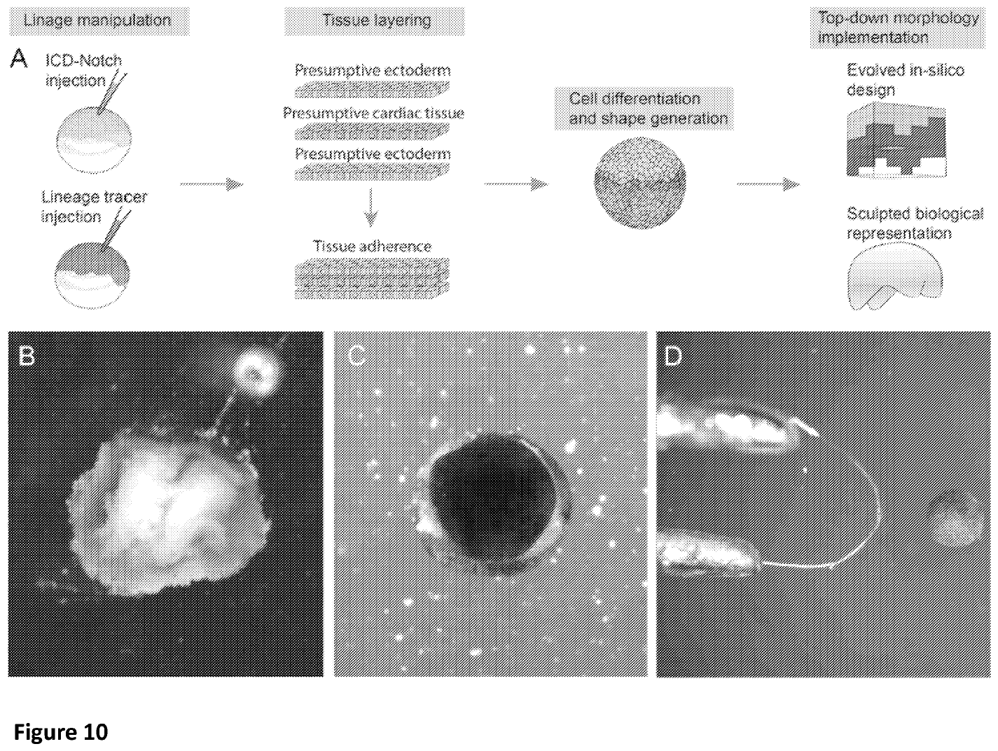

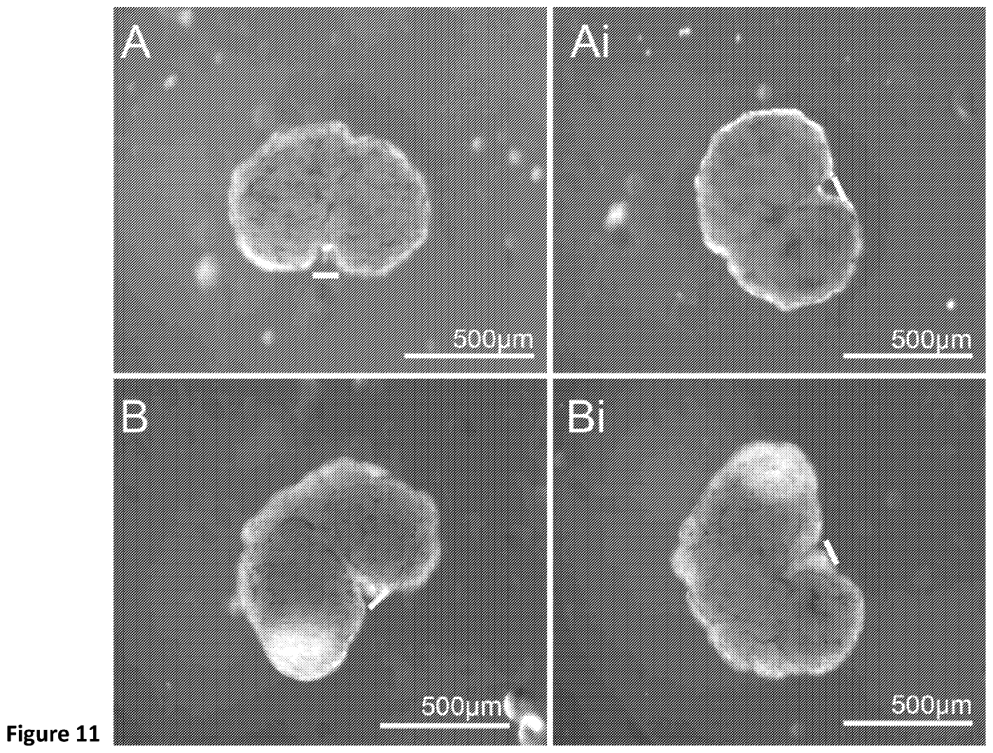

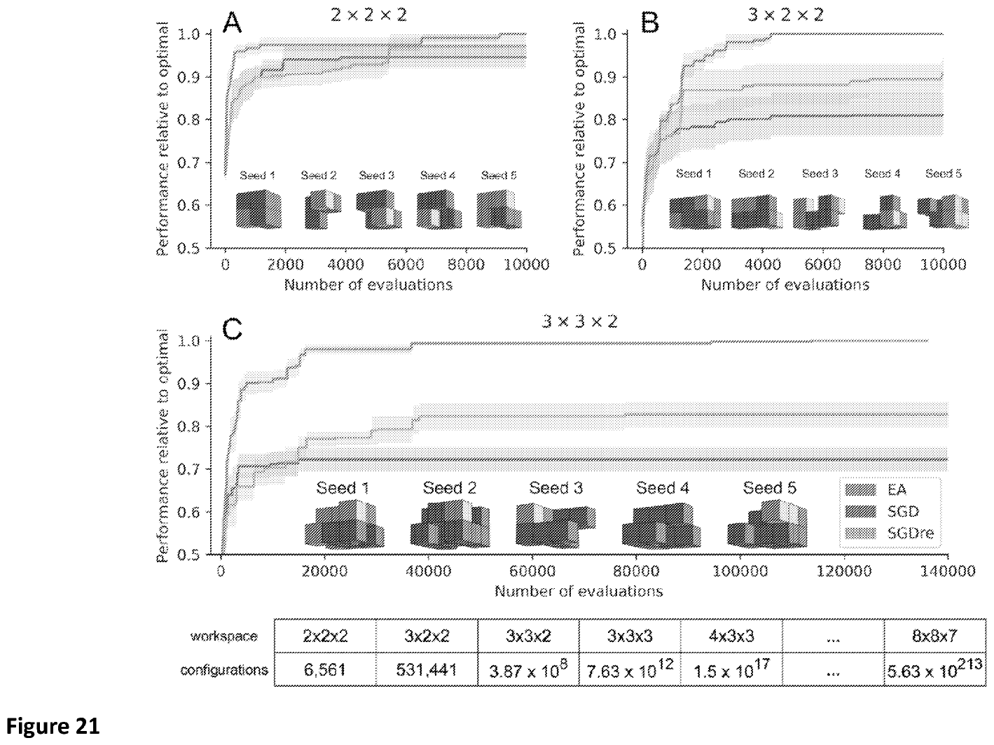

The surviving noise-resistant designs are then passed through a build filter (See Example, FIG. 8) which removes designs that are not suitable for the current build method (See Example, FIG. 10) or unlikely to scale to more complex tasks in future deployments. The manufacturability of a design depends on the minimal concavity size that will persist in aggregations of developing stem cells, which tend to close small gaps in their collective geometry (See Example, FIG. 11). The scalability of a design depends on its proportion of passive tissue, which provides space for future organ systems or payloads. (See Example, FIG. 21).

The designs that successfully pass through the build filter are then built out of living tissues. Pluripotent stem cells are first harvested from blastula stage Xenopus laevis embryos, dissociated, and pooled to achieve the desired number of cells. Following an incubation period, the aggregated tissue is then manually shaped by subtraction using a combination of microsurgery forceps and a 13-μm wire tip cautery electrode, producing a biological approximation of the simulated design. Further, contractile tissue can be layered into the organism through the harvesting and embedding of Xenopus cardiac progenitor cells, an embryonically derived cell type which naturally develops into cardiomyocytes (heart muscle) and produces contractile waves at specific locations in the resultant shaped form (See Example, FIG. 10).

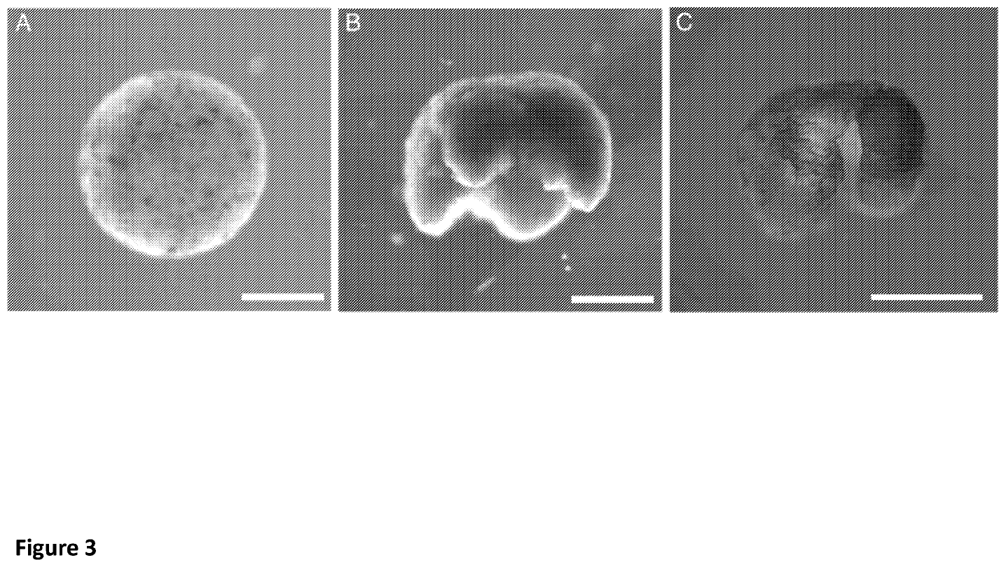

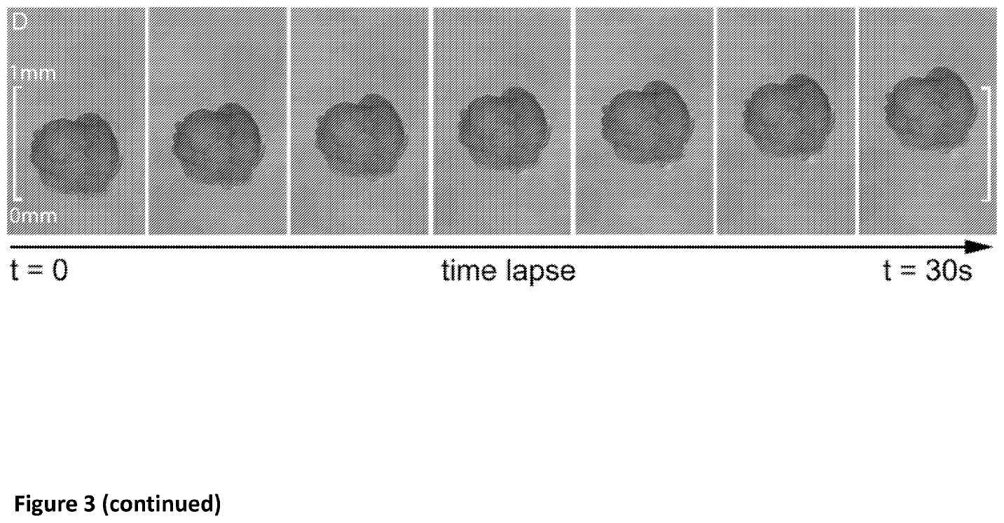

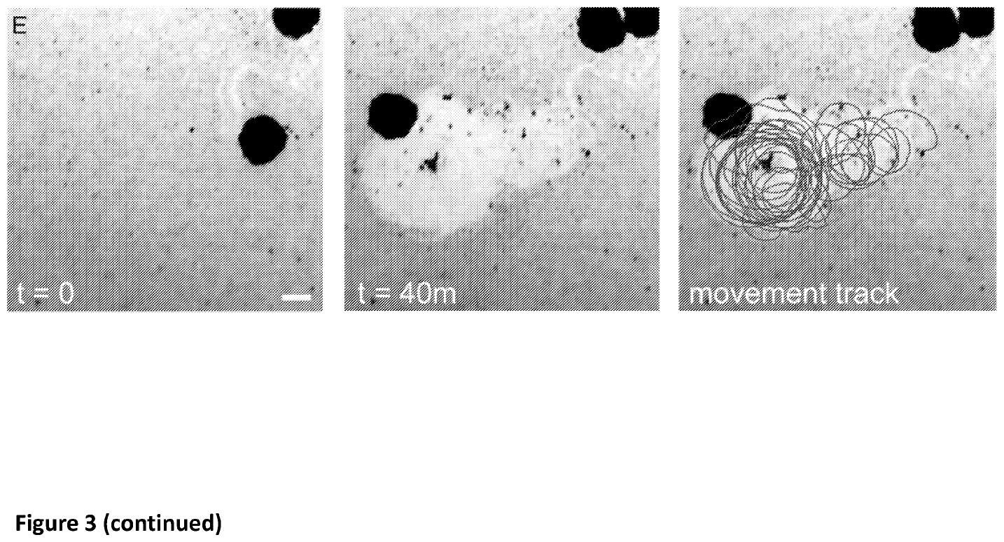

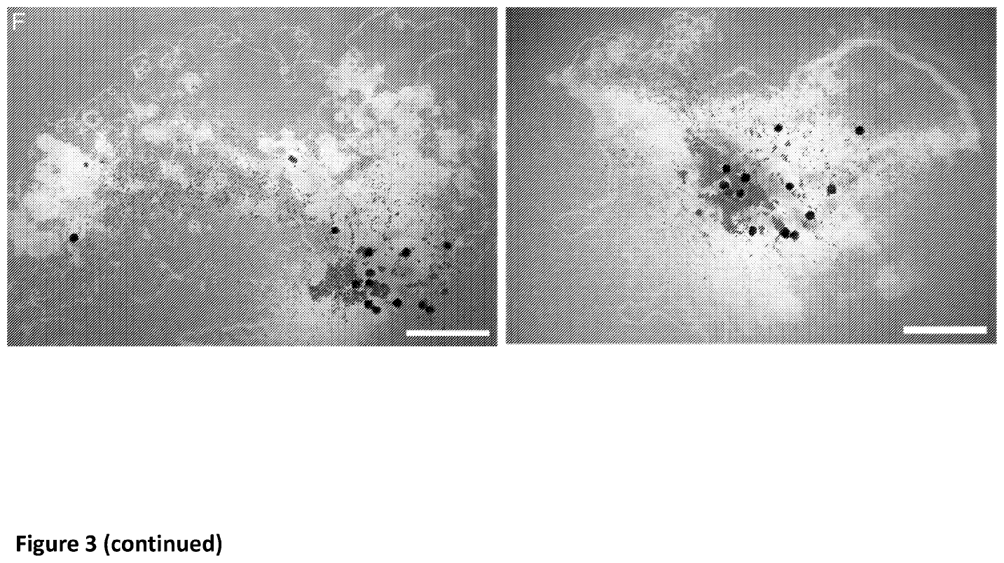

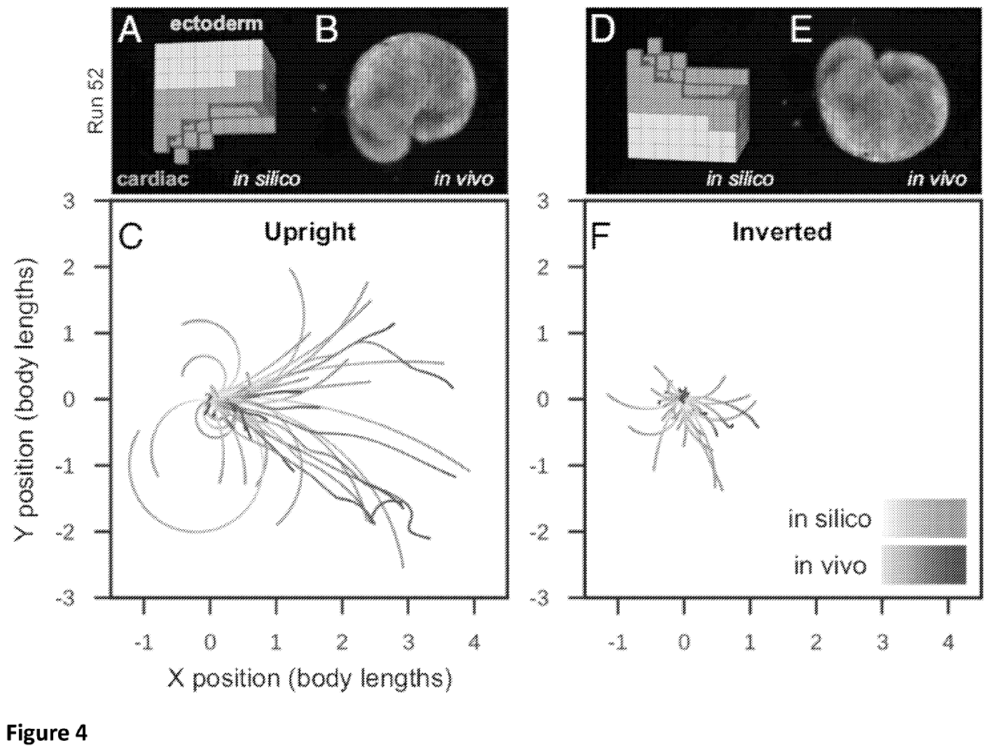

The final product of this procedure is a living, 3D approximation of the evolved design, which possesses the ability to self-locomote and explore an aqueous environment for a period of days or weeks without additional nutrients. These organisms are then deployed into their physical environment, and resultant behavior, if any, is observed (FIG. 3). Behaviors are then compared against those predicted by their simulated counterparts to determine whether or how well behaviors transferred from silico to vivo (FIG. 4).

After several organisms have been deployed and observed, it is likely that they exhibit varying amounts of the desired behavior. Common patterns among the successful systems are distilled down into constraints and supplied back to the evolutionary algorithm, which now evolves designs that are not just performant but also conform to the constraints. (See Example, section 6). This increases the success likelihood of subsequent design-to-deployment attempts. Reconfigurable organisms were evolved to exhibit four different behaviors: locomotion, object manipulation, object transport, and collective behavior (See Example, section 10). To achieve this, the pipeline was employed four times.

Locomotion. To obtain a diverse population of designs, 100 independent trials of the evolutionary algorithm were conducted (FIG. 2A-C), each starting from a different set of initial random designs. During each trial, designs were selected based on net displacement achieved during a 10-s period (with randomized, phase-modulated contraction, cycling at 2 Hz). Additional selection pressures were applied to maintain diversity by inducing competition within and between unique genetic lineages within each trial (19), yielding unique ecological dynamics (See Example, section 5). The most fit designs at the end of each trial were extracted (FIG. 1A) and passed through the robustness and build filters (See Example, FIG. 8). During this filtering process, buildable and scalable designs that retain rapid locomotion during random perturbations are selected for manufacture (FIG. 3 and See Example, FIG. 10).

Cilia, which produce locomotion through metachronal waves (the generation of sequential and directional propagating waves, as opposed to synchronized beating), were not modeled in silico and were suppressed in vivo through embryonic microinjection of mRNA transcribing the Notch intracellular domain (Notch ICD) (20). Thus, any displacement results from contractile cardiac muscle tissue that pushes against the surface of the dish. This simplifies the simulation and its comparison to the realized organism. Trajectories of deciliated designs are compared in silico and in vivo, in two orientations (upright and inverted 180° about the transverse plane) thus isolating the impact of the designed morphology on the difference between predicted and realized behavior. For at least one design, the data suggest that the desired behavior successfully transferred when it was upright but not when inverted (FIG. 4). More specifically, the upright organisms' direction of movement matched that of the in silico design under random perturbations (P<0.01; details in See Example, section 9), and inverting the design significantly reduced its net displacement both in silico (P<0.001) and in vivo (P<0.0001). This suggests that successful transference did not result by chance but rather was due to the design itself.

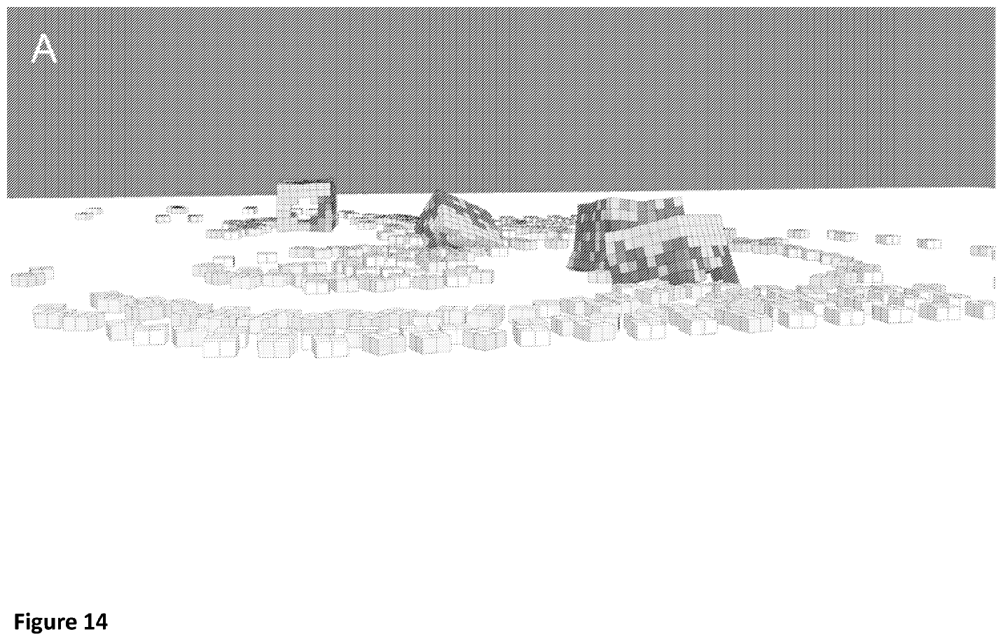

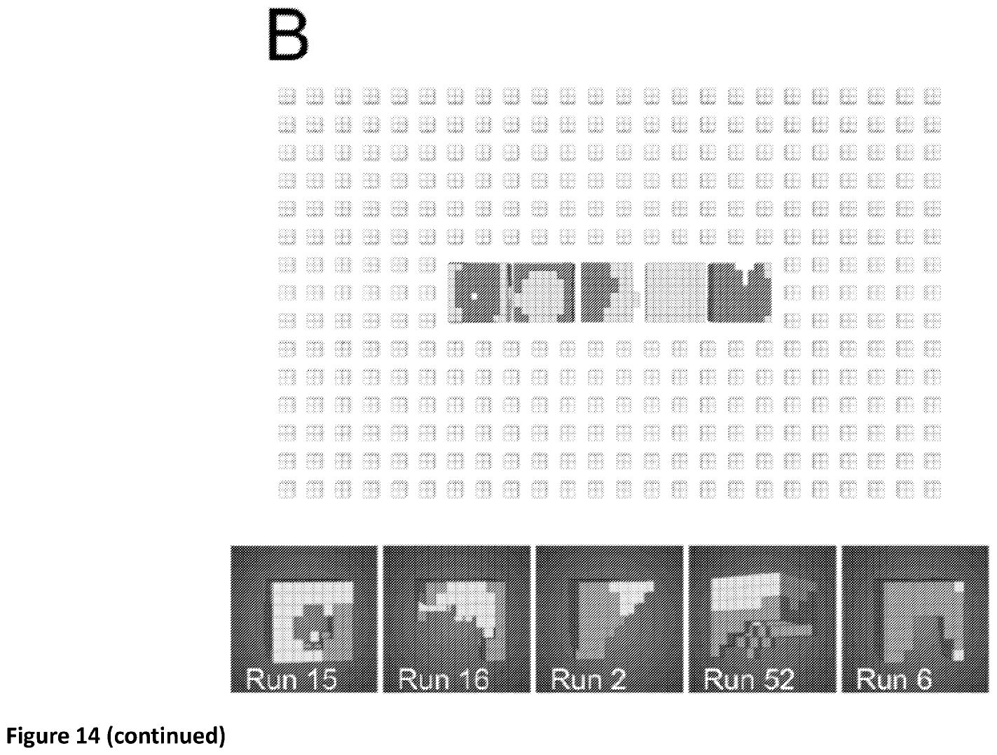

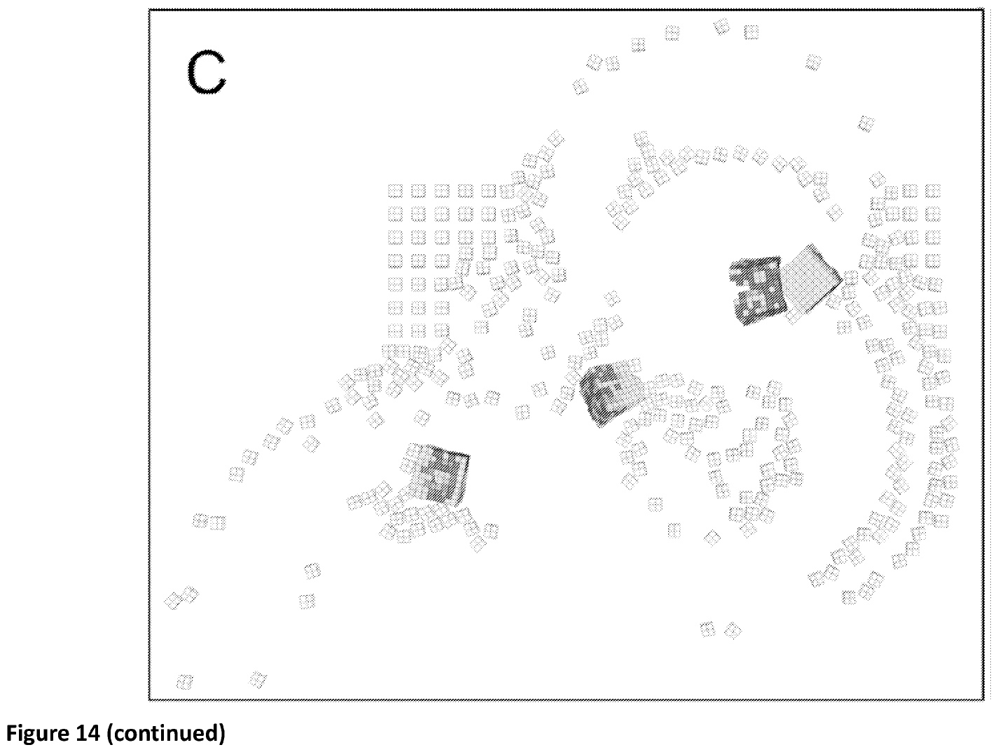

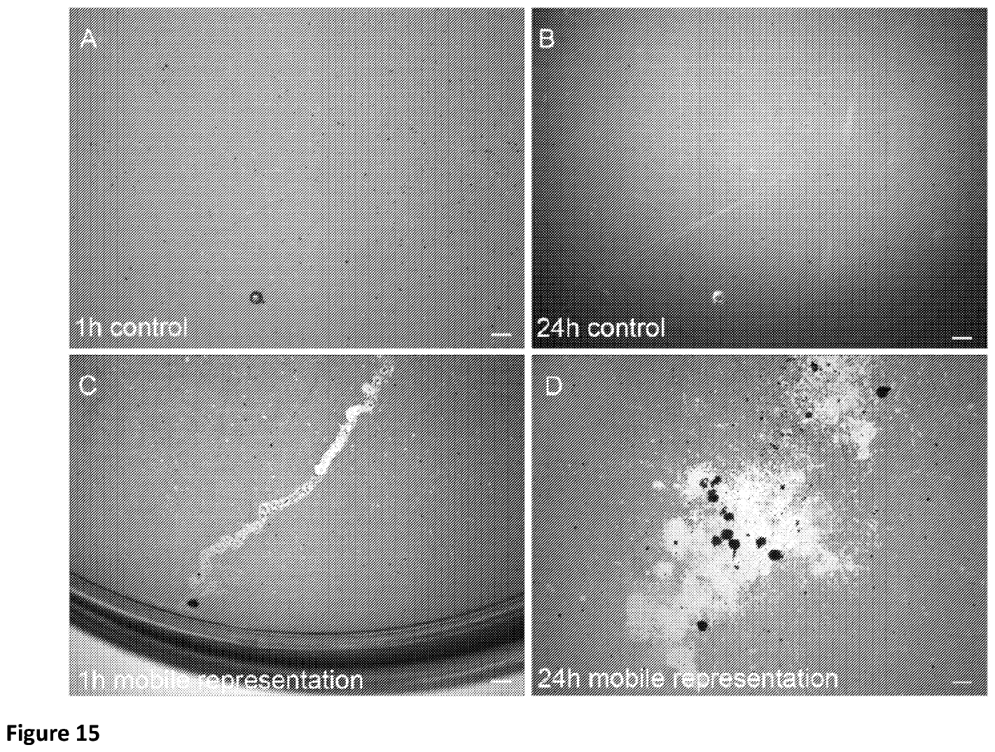

Object Manipulation. When the environment is strewn with particulate matter, motile designs spontaneously aggregate the external objects both in silico. (See Example, FIG. 14) and in vivo (FIG. 3F and See Example, FIG. 15). More precise object manipulation can be selected for an explicit goal, such as specifying target areas from which debris should be cleared, or target objects to discard. The latter goal was implemented and primitive end-effectors evolved in simulation (See Example, FIG. 16).

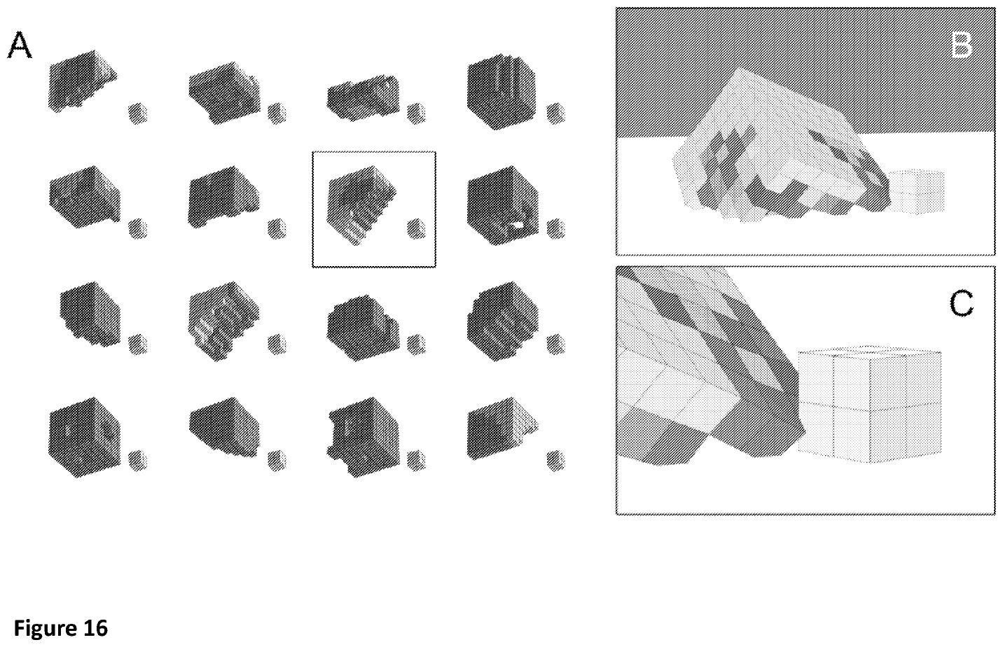

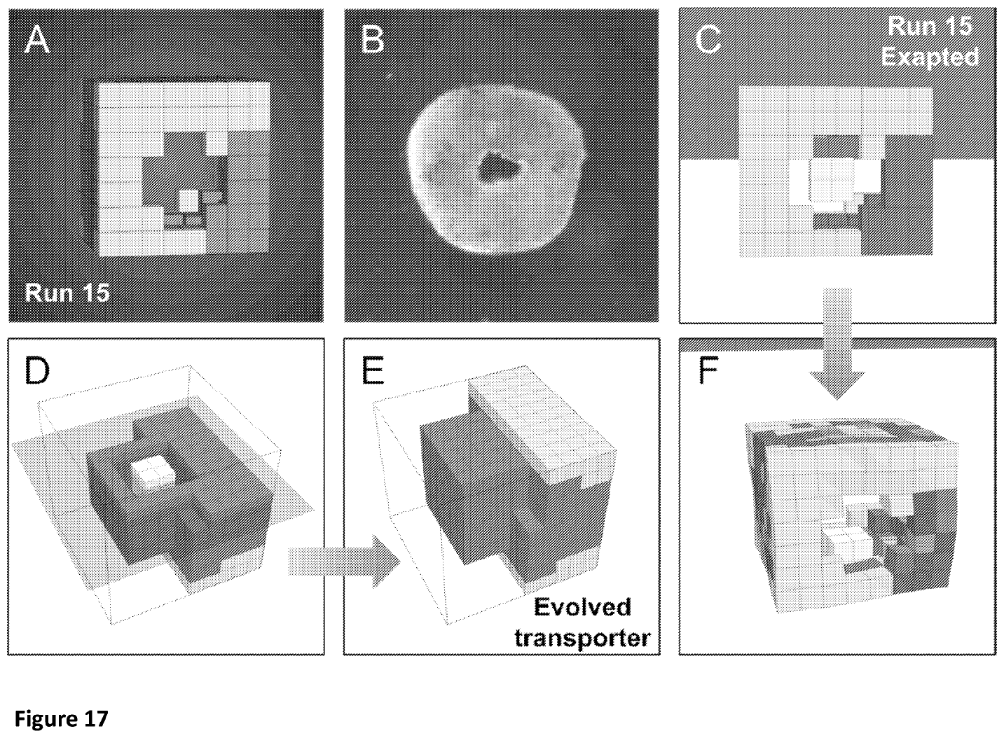

Object Transport. Some designs evolved for displacement reduced hydrodynamic drag (See Example, section S6) via a hole through the center of their transverse plane. This more complex topology was realized in vivo (See Example, FIG. 17) but was not layered with contractile tissue. In simulation, this emergent feature can be exapted as a pouch to store and transport objects. In a subsequent round of evolution, pouches were explicitly incorporated as a design constraint, and the new goal of maximizing the distance of the carried object was employed. This yielded evolved object transport in silico (See Example, FIG. 17).

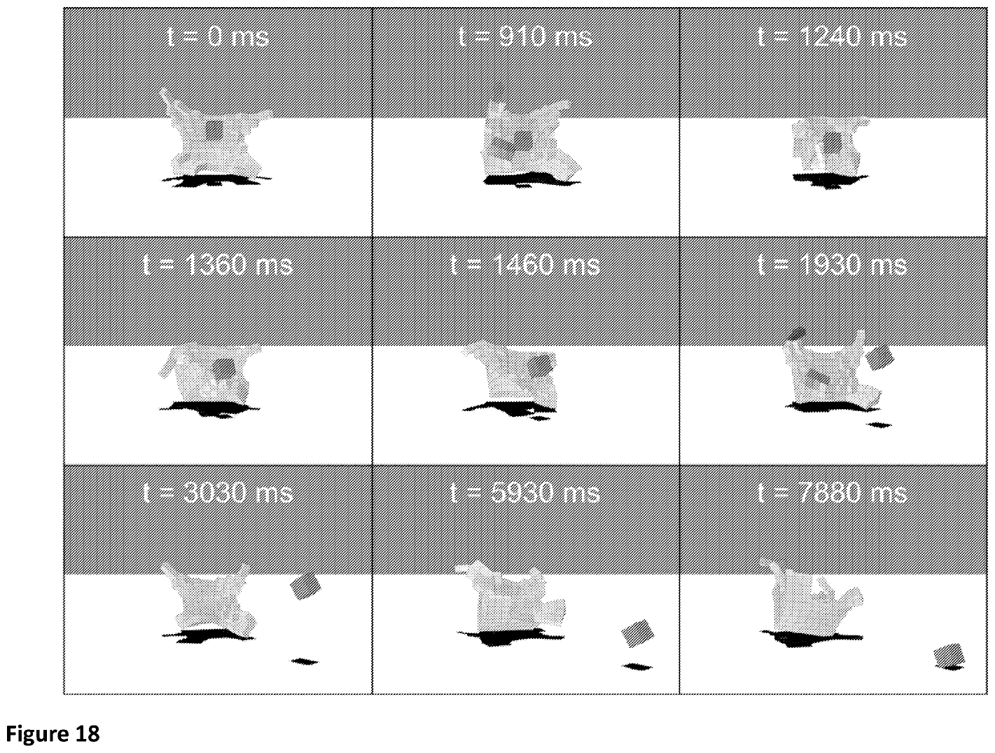

Collective Behavior. Multiple designs can be placed in the same environment, yielding collective behavior (21) (See Example, FIGS. 14 and 15). Several such behaviors predicted in silico were observed in vivo. For instance, two designs often collide, form a temporary mechanical bond, and orbit about each other for several revolutions before detaching along tangential trajectories (See Example, FIG. 18). This phenomenon is more pronounced when cilia are not inhibited on the organisms: individuals frequently become entangled with their neighbors, often changing partners across an observation (FIG. 3F and See Example, FIG. 15).

### DISCUSSION

Although simulation and design of rigid structures and machines has been possible for some time, only recently has it become computationally tractable to simulate the combined behavior of arbitrary aggregates of soft components with differing material and actuation properties (22). As shown here, such fine-grained simulations can be embedded in evolutionary search methods to discover designs that can be instantiated in biological rather than artificial materials.

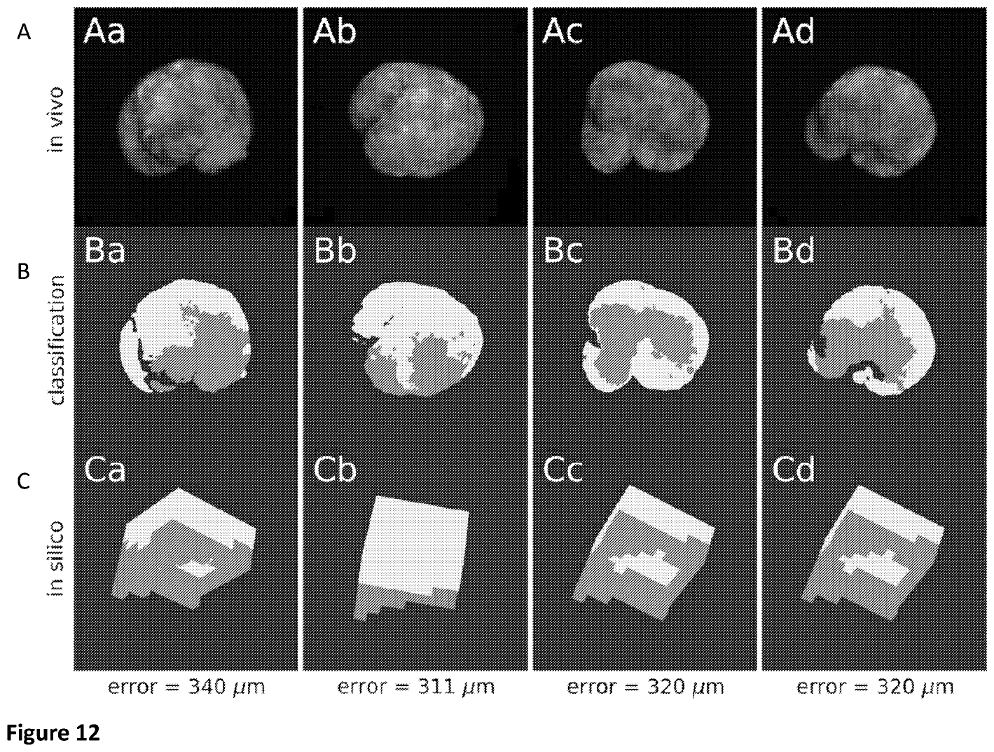

The resulting organisms embodied not only the structure (See Example, FIG. 12) of evolved in silico designs but also their behavior (FIG. 4), despite modeling cardiomyocyte temporal coordination as random noise. As a side effect of selection pressure for locomotion, derandomizing morphologies evolved: evolutionary improvement occurred through changes in overall shape, and distribution of the passive and contractile cells, to collectively derandomize the global movement produced by the random actuation. In biology, such robustness to random noise is ubiquitous; one example is the ability of many species to adapt to wide ranges of diversity in cell size and number as starting points in their embryogenesis (23).

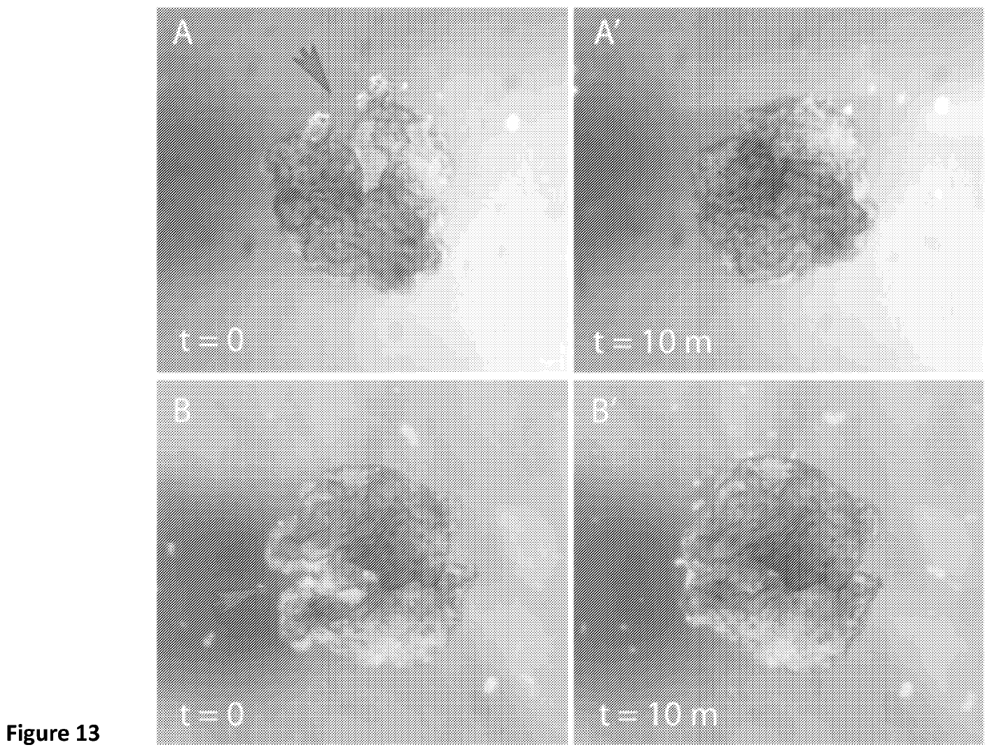

The behavioral competence of individual cells, and the propensity of cells to cooperate in groups, facilitate functional morphogenesis in novel circumstances. The lifeforms presented here, despite lacking nervous systems, following novel developmental trajectories, and being composed of materials from different tissues, nevertheless possess these self-organizing properties. These properties synergize with and support the behavior they were designed to exhibit. For instance, although signaling between cardiomyocytes was not enforced, emergent spontaneous coordination among the cardiac muscle cells produced coherent, phase-matched contractions which aided locomotion in the physically realized designs. Also, some of the designs, when combined, spontaneously and collectively aggregate detritus littered within their shared environment (FIG. 3F and See Example, FIG. 15). Finally, reconfigurable organisms not only self-maintain their externally imposed configuration, but they also self-repair in the face of damage, such as automatically closing lacerations (See Example, FIG. 13). Such spontaneous behavior cannot be expected from machines built with artificial materials unless that behavior was explicitly selected for during the design process (24).

This approach admits future generalization and automation because the generator-and-filter architecture enables modular addition, removal, or reorganization of elements in the pipeline for rapid design and deployment of new living systems for new tasks in new domains. For instance, a filter could be added which preemptively steers the evolutionary algorithm away from portions of the design space known to contain designs that cannot be realized physically (25). Or, inspired by the hierarchical organization of deep neural networks (26), individual designs output by one generator could become the building blocks input to the next generator, thus enabling hierarchical design and reuse of cellular assemblies, and assemblies of assemblies.

Beyond the applications reported here, the generality of this approach is as of yet unknown. But, advances in machine learning, soft body simulation, and bioprinting are likely to broaden the potential applications to which it may be put in the future. Applications could be numerous, given the ease of misexpressing novel proteins and synthetic biology pathways and computational circuits in Xenopus cells (27). Given their nontoxicity and selflimiting lifespan, they could serve as a novel vehicle for intelligent drug delivery (28) or internal surgery (29). If equipped to express signaling circuits and proteins for enzymatic, sensory (receptor), and mechanical deformation functions, they could seek out and digest toxic or waste products, or identify molecules of interest in environments physically inaccessible to robots. If equipped with reproductive systems (by exploiting endogenous regenerative mechanisms such as occurs in planarian fissioning), they may be capable of doing so at scale. In biomedical settings, one could envision such biobots (made from the patient's own cells) removing plaque from artery walls, identifying cancer, or settling down to differentiate or control events in locations of disease. A beneficial safety feature of such constructions is that in the absence of specific metabolic engineering, they have a naturally limited lifespan.

These methods, reagents, and data extend the breadth of model organisms available for study by designing living systems that are as orthogonal as possible to existing species, yet capable of being built from existing cell types. By enabling a computationally guided interplay between emergent and designed processes, this platform facilitates studies of the relationship between genomes (in our case, wild-type X. laevis), the resulting body plan, and its behaviors in diverse environments. Thus, reconfigurable organisms could serve as a unique model system facilitating work in the evolution of multicellularity, exobiology, artificial life, basal cognition, and regenerative medicine. If equipped with electrically active cells and selected for cognitive or computational functions (30), such designed systems may similarly broaden our understanding of how intelligence can be instantiated in living as well as nonliving systems.

Materials and Methods

Evolutionary Design. Designs (See Example, section 2) were evolved inside a physics engine (See Example, section 3) as reconfigurable aggregations of passive and contractile voxels (FIG. 1). On the first pass through the pipeline using the goal behavior of locomotion, we simulated designs on land and allowed the evolutionary process to finely tune their actuation. This resulted in highly performant but nontransferable designs (See Example, FIG. 6) with powerful, bounding gaits that are not obtainable in vivo with the current build method (See Example, section 8). These gaits were characterized by timeframes (on average, 47% of the gait cycle) in which no part of the in silico design was in contact with the simulated ground plane. In vivo, however, the deciliated organisms always kept part of their ventral surfaces in contact with the surface of the dish due to negative buoyancy.

These discrepancies were rectified by adding constraints into the pipeline in the form of adjustments to environmental and actuation settings, which were altered as follows. On the second pass, the fidelity of the simulated environment was increased by incorporating first-order hydrodynamics: the modified environment consisted of an infinite plane submerged in water, which was approximated by decreasing the coefficient of gravitational acceleration (increasing buoyancy) and applying a drag force to each voxel face on the design's surface (See Example, section 6).

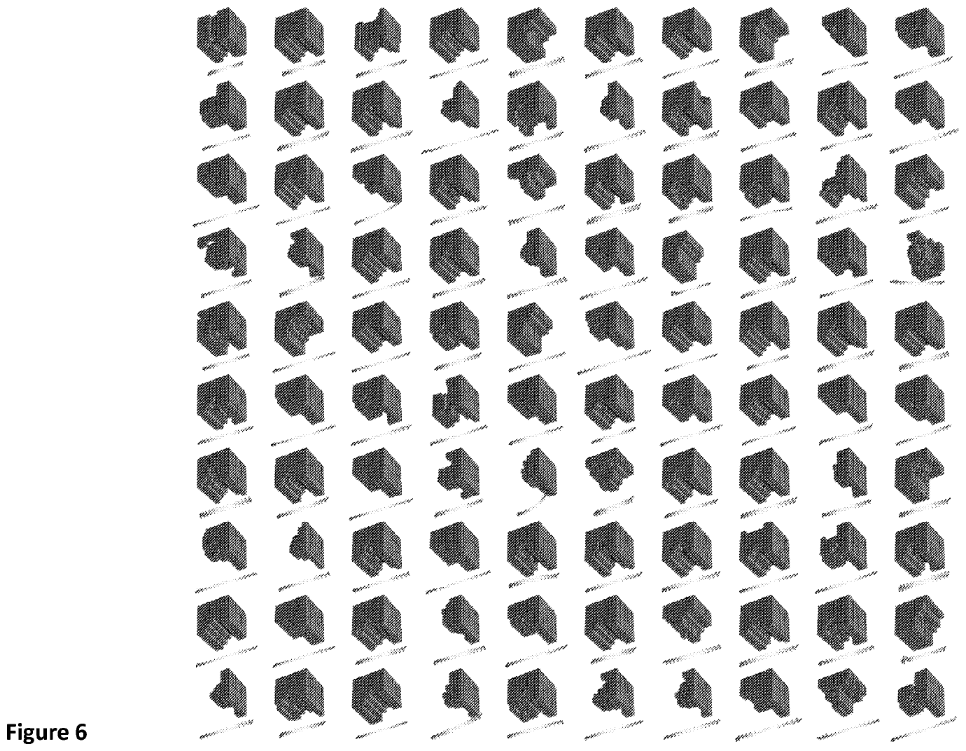

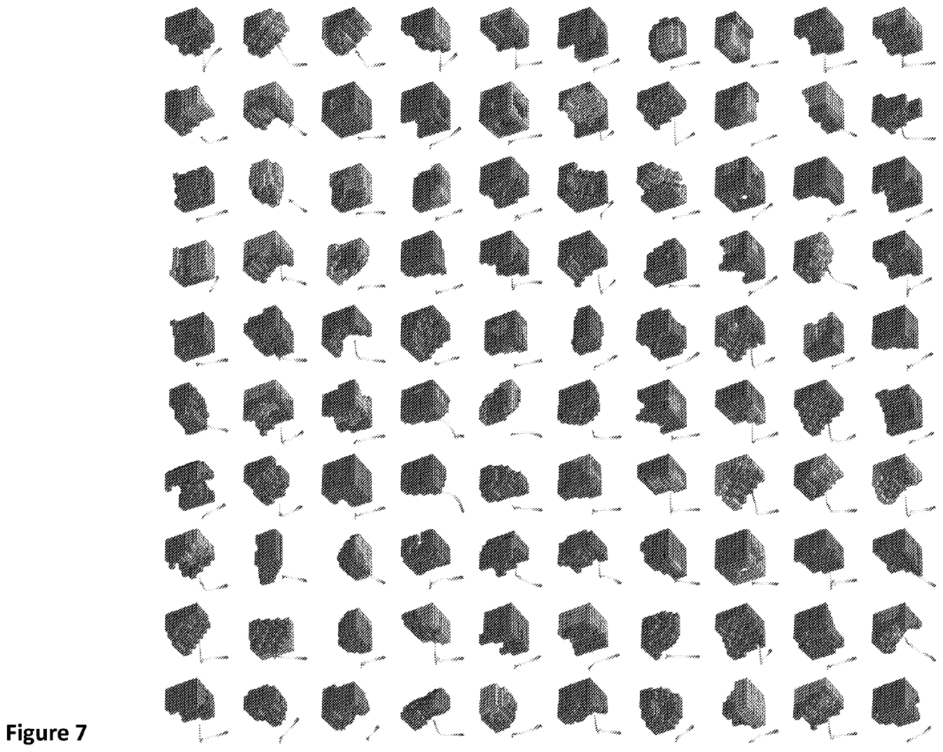

Secondly, actuation was randomized: contractile cells were revised to have random phase offsets from a central pattern generator (a sine wave with frequency 2 Hz). More specifically, each voxel of a randomly configured design (one of which was injected into the population at each generation; See Example, section 5) was assigned a random phase offset, which was held fixed in its descendants (the entire clade). Mutations switched each voxel to be either present or absent, and, if present, either passive or active (contractile), but the original phase offset, at every location in the workspace, was hardcoded. This reduced the dependence on precisely timed excitation, and promoted the discovery of more robust mechanical structures (See Example, FIG. 7).

The behavior of designs generated on the second pass better matched the behavior of the actual living systems: on average, designs were in contact with the ground plane for 93.3% of their evaluation period, compared to just 52.7% on the first pass (See Example, section 6).

Robustness Filter. The most performant designs (FIG. 1A) were sorted by their robustness to random perturbations in their actuation. Phase offsets stored in the genotype were mutated by adding a number that was drawn randomly from a normal distribution with mean zero and SD s=0.4π (which is 40% of the −π/2 to π/2 range of valid phase-offset values). This hyperparameter was selected to be large enough to scramble the original phase-offset value without being so large as to push all mutations up against the ±π/2 bounds. Designs that maintained the highest average performance across this actuation noise were passed, one by one, in order of their robustness ranking, to the build filter.

Build Filter. The most robust designs are evaluated by their manufacturability under the current build method, which layers contiguous tissue regions sequentially (See Example, FIG. 10). The minimal concavity was examined by producing organisms with progressively smaller shape deformations, then determining which persist across the lifespan of the organism, and which close due to tissue contraction, leading to loss of concavity. Preliminary work determined that concavities with a width of 100 μm or greater (12% of total body length) produced stable long-term deformations suitable for biological building (See Example, FIG. 11).

Additionally, the build filter removes designs that are more than 50% muscle, in order to reserve sufficient design space to add specialized cells for purposes other than locomotion, including sensory input, metabolism, memory, biosensors, etc. Also, contractile tissue incurs a much higher metabolic cost compared to nonmuscle tissue (the human heart consumes −1 mM ATP per second; ref. 31). Thus, limiting this tissue type increases the total lifetime of transferred designs. The most robust designs that satisfy these selection criteria (See Example, FIG. 8) are passed through the build filter to the next stage of the pipeline: the realizability generator.

Realizability Generator. Reconfigurable organisms were created using Xenopus embryos as donor tissue under methods approved by the Institutional Animal Care and Use Committee and Tufts University Department of Laboratory Animal Medicine under protocol number M2017-53. Fertilized X. laevis eggs were reared in a 0.1×, pH 7.8, Marc's Modified Ringers solution (MMR) using standard protocols and staged according to Nieuwkoop and Faber (32, 33). For shaping experiments, animal caps were manually cut at St. 9 using surgery forceps (Dumont, 11241-30 #4) and transferred to calcium- and magnesium-free medium for 5 min (50.3 mM NaCl, 0.7 mMKCl, 9.2 mM Na₂HPO₄, 0.9 mM KH₂PO₄, 2.4 mM NaHCO₃, 1.0 mM edetic acid [EDTA], pH 7.3). The outer ectoderm layer was manually removed and discarded, while the inner layer was agitated until fully dissociated (cells are this stage are largely pluripotent, but differentiate into ectoderm without further intervention). Material from five animal caps was pooled and transferred to a welled dish containing 0.75×MMR. After 24 h at 14° C., the spherical reaggregate was moved to a clean 1% agarose-coated dish containing 10 mL 0.75×MMR and 5 gentamicin (ThermoFisher Scientific, 15710072). Forty-eight hours after tissue reaggregation the resulting tissue (now fated to become specific epidermal cell lineages including ionocytes, small secretory cells, and goblet cells), was shaped using a combination of microsurgery forceps and a MC-2010 microcautery instrument with 13-μm wire electrodes (Protech International Inc., MC-2010, 13-Y1 wire tip cautery electrode). Tissue was reshaped as necessary for 3 h to create the desired anatomical outcome, after which it was moved to a clean 1% agarose-coated dish containing 10 mL 0.75×MMR and 5 μl gentamicin and raised at 14° C.

For contractile movement experiments, cohorts of Xenopus embryos were microinjected with one of two synthetic mRNAs at the four-cell stage using standard protocols (32). mRNA for the fluorescent lineage tracer tdTomato (34) and the multiciliated cell inhibitor Notch ICD (20, 35) was synthesized using mMESSAGE transcription kits (ThermoFisher Scientific, AM1340). Injections were performed in 3% Ficoll solution using a pulled capillary to deliver 370 pg of mRNA for each transcript to all four cells. tdTomato microinjected embryos were reared for at 22° C. while Notch ICD injected embryos were reared at 14° C. Twenty-four hours after injection, stage 10 Notch ICD injected embryos were moved to a 1% agarose-coated Petri dish containing 0.75×MMR, and animal caps were manually cut using surgery forceps as above. In addition, stage 23-24 tdTomato injected embryos were transferred to the same dish and the presumptive heart field was excised with the outer layer of ectoderm then removed and discarded. Presumptive heart tissue was then placed between two Notch ICD injected animal caps, and the three layers were allowed to heal for 1 h at 22° C. Following healing, the tissue was moved to clean 1% agarose-coated dish containing 10 mL 0.75×MMR and 5 μl gentamicin and raised at 14° C. For shaping, resultant tissue was sculpted as above using a combination of microsurgery forceps and a MC-2010 microcautery instrument.

Transferability Filter. All samples were imaged live in 0.75×MMR at 20° C. using a Nikon SMZ-1500 microscope equipped with both top and substage illumination. Still images were captured on a QImaging Retiga 2000R CCD camera and videos were captured using a Sony IMX234 at a sample rate of 30 frames per second. XY movement tracks were extracted for each run using Noldus Ethovision 14 software, and smoothed using a one-dimensional Gaussian filter (See Example, section 9.1). The tdTomato lineage tracer was imaged using a standard tetramethylrhodamine isothiocyanate (TRITC) filter cube and fluorescent light source to verify cardiac muscle cell location, and GFPIII signal was imaged with a standard fluorescein isothiocyanate (FITC) filter cube to verify epidermal cell location (See Example, section 9.2).

Data Availability. The source code necessary for reproducing the computational results reported in this paper can be found at GitHub (36).

### REFERENCES

1. D. J. Blackiston, M. Levin, Ectopic eyes outside the head in Xenopus tadpoles provide sensory data for light-mediated learning. J. Exp. Biol. 216, 1031-1040 (2013).
2. L. N. Vandenberg, D. S. Adams, M. Levin, Normalized shape and location of perturbed craniofacial structures in the Xenopus tadpole reveal an innate ability to achieve correct morphology. Dev. Dyn. 241, 863-878 (2012).
3. R. D. Kamm et al., Perspective: The promise of multi-cellular engineered living systems. APL Bioeng 2, 040901 (2018).
4. C. A. Hutchison, III et al., Design and synthesis of a minimal bacterial genome. Science 351, aad6253 (2016).
5. Y. Sasai, M. Eiraku, H. Suga, In vitro organogenesis in three dimensions: Selforganising stem cells. Development 139, 4111-4121 (2012).
6. S. J. Park et al., Phototactic guidance of a tissue-engineered soft-robotic ray. Science 353, 158-162 (2016).
7. M. D. Tang-Schomer et al., Bioengineered functional brain-like cortical tissue. Proc. Natl. Acad. Sci. U.S.A. 111, 13811-13816 (2014).
8. J. C. Nawroth et al., A tissue-engineered jellyfish with biomimetic propulsion. Nat. Biotechnol. 30, 792-797 (2012).
9. K. Sims, Evolving 3D morphology and behavior by competition. Artif. Life 1, 353-372 (1994).
10. N. Cheney, J. Bongard, V. SunSpiral, H. Lipson, Scalable co-optimization of morphology and control in embodied machines. J. R. Soc. Interface 15, 20170937 (2018).
11. H. Lipson, J. B. Pollack, Automatic design and manufacture of robotic lifeforms. Nature 406, 974-978 (2000).
12. J. Bongard, V. Zykov, H. Lipson, Resilient machines through continuous self-modeling. Science 314, 1118-1121 (2006).
13. D. Cellucci, R. MacCurdy, H. Lipson, S. Risi, 1D printing of recyclable robots. IEEE Robot. Autom. Lett. 2, 1964-1971 (2017).
14. D. J. Munk, G. A. Vio, G. P. Steven, Topology and shape optimization methods using evolutionary algorithms: A review. Struct. Multidiscipl. Optim. 52, 613-631 (2015).
15. R. V. Devi, S. S. Sathya, M. S. Coumar, Evolutionary algorithms for de novo drug design—A survey. Appl. Soft Comput. 27, 543-552 (2015).
16. M. D. Huntington, L. J. Lauhon, T. W. Odom, Subwavelength lattice optics by evolutionary design. Nano Lett. 14, 7195-7200 (2014).
17. C. T. Mueller, J. A. Ochsendorf, Combining structural performance and designer preferences in evolutionary design space exploration. Autom. Constr. 52, 70-82 (2015).
18. N. Jakobi, Evolutionary robotics and the radical envelope-of-noise hypothesis. Adapt. Behav. 6, 325-368 (1997).
19. M. Schmidt, H. Lipson, “Age-fitness pareto optimization” in Genetic Programming Theory and Practice VIII (Springer, 2011), pp. 129-146.
20. G. A. Deblandre, D. A. Wettstein, N. Koyano-Nakagawa, C. Kintner, A two-step mechanism generates the spacing pattern of the ciliated cells in the skin of Xenopus embryos. Development 126, 4715-4728 (1999).
21. J. Werfel, K. Petersen, R. Nagpal, Designing collective behavior in a termite-inspired robot construction team. Science 343, 754-758 (2014).
22. J. Hiller, H. Lipson, Dynamic simulation of soft multimaterial 3d-printed objects. Soft Robot. 1, 88-101 (2014).
23. J. Cooke, Scale of body pattern adjusts to available cell number in amphibian embryos. Nature 290, 775-778 (1981).
24. S. Kriegman, S. Walker, D. Shah, M. Levin, R. Kramer-Bottiglio, J. Bongard, Automated shapeshifting for function recovery in damaged robots. Proc. Rob. Sci. Syst. (2019).
25. S. Koos, J. B. Mouret, S. Doncieux, The transferability approach: Crossing the reality gap in evolutionary robotics. IEEE Trans. Evol. Comput. 17, 122-145 (2013).
26. M. D. Zeiler, R. Fergus, “Visualizing and understanding convolutional networks” in Proceedings of the European Conference on Computer Vision, D. Fleet, T. Pajdla,
B. Schiele, T. Tuytelaars, Eds. (Springer, 2014), pp. 818-833.
27. S. Toda, L. R. Blauch, S. K. Y. Tang, L. Morsut, W. A. Lim, Programming self-organizing multicellular structures with synthetic cell-cell signaling. Science 361, 156-162 (2018).
28. D. Patra et al., Intelligent, self-powered, drug delivery systems. Nanoscale 5, 1273-1283 (2013).
29. J. Li, B. Esteban-Fernandez de Avila, W. Gao, L. Zhang, J. Wang, Micro/nanorobots for biomedicine: Delivery, surgery, sensing, and detoxification. Sci. Rob. 2, eaam6431 (2017).
30. F. Baluška, M. Levin, On having no head: Cognition throughout biological systems. Front. Psychol. 7, 902 (2016).
31. J. Piquereau, R. Ventura-Clapier, Maturation of cardiac energy metabolism during perinatal development. Front. Physiol. 9, 959 (2018).
32. P. D. Nieuwkoop, J. Faber, Normal Table of Xenopus laevis (Daudin) (North Holland Publishing Co., Amsterdam, 1956).
33. H. L. Sive, R. M. Grainger, R. M. Harland, Early Development of Xenopus laevis (CSHL Press, 2000).
34. C. Waldner, M. Roose, G. U. Ryffel, Red fluorescent Xenopus laevis: A new tool for grafting analysis. BMC Dev. Biol. 9, 37 (2009).
35. C. W. Beck, J. M. Slack, A developmental pathway controlling outgrowth of the Xenopus tail bud. Development 126, 1611-1620 (1999).
36. S. Kriegman, D. Blackiston, M. Levin, J. Bongard, Data from “A scalable pipeline for designing reconfigurable organisms.” GitHub. https://github.com/skriegman/reconfigurable_organisms. Deposited 2 Oct. 2019.

Supplemental Information

1. The source code: github.com/skriegman/reconfigurable_organisms

2. The design space.

This subsection gives bounds on the number of designs that can be built in a voxel-based workspace.

A distinct configuration of exactly N congruent cubes (voxels) connected face-to-face is known as a polycube. With N=2, there is just a single configuration: a 2-by-1 column (or dicube). But there are two perpendicular rotations: if one cube is resting on the ground, we can add the second on top or on the side.

Rotations of the same configuration can be treated as equivalent, or not. Polycubes are called “real” if equivalent under rotation, and “fixed” if not. With N=3, there are two real polycubes (tricubes), and 15 fixed. With N=4, there are eight real (tetracubes), and 86 fixed. As N increases, the number of polycubes grows exponentially. With N=16 cubes, for instance, there are on the order of 10 10 real polycubes and 10 12 fixed polycubes (36).

But this assumes that all cubes are identical. Here, the design space consists of polycubes composed of two cube types (passive and contractile), with any N, that fit inside an 8×8×7 bounding box. Many polycubes with N>7 won't fit inside this workspace. And with N=8×8×7=448, there is only one polycube geometry that fits, but there are still 2⁴⁴⁸=7.27×10¹³⁴ possible combinations of passive and contractile voxels that could be used to build the polycube.

Because the designs here are functional polycubes, their uniqueness depends on the behavioral goal. For locomotion in any direction away from the origin on a surface plane, designs can yield different behavior when inverted (FIG. 4), but the same behavior when the design is rotated about the vertical axis (just facing a different direction). Because there are three tissue options (none, passive, and contractile) at each point in the workspace, there are 3⁴⁴⁸=5.63×10²¹³ possible configurations, though some are isomorphic translations after reducing to a single (the largest) polycube.

3. The physics engine.

This subsection briefly describes the physics of the in silico environment. For more details, see (22).

Experiments were performed using the voxel-based physics engine Voxelyze (22). Voxels were connected to each other on a regular grid to form a contiguous geometry: a polycube. Interactions between adjacent voxels were modeled as flexible beams (critically damped; zeta=1) according to Euler-Bernoulli beam theory. A Coulomb friction model was applied to voxels in contact with the ground surface plane. Volumetric actuation resulting from contractile cells was simulated by oscillating the rest length between adjacent actuating voxels, in all three dimensions, when computing the elastic force between them. Additionally, a collision detection system ensured that the organisms did not self-penetrate. If a pair of surface voxels are detected to collide (intersect), a temporary beam (underdamped; zeta=0.8) was constructed between the two until the collision is resolved. A time step of 0.0032 seconds was used for numerical integration. Each design was allowed to settle under gravity for 312 timesteps (one second) before an evaluation period of 3125 timesteps (10 seconds), resulting in a total simulation time of 11 seconds.

4. The encoding.

This subsection defines the genetic search space evolution operates in. Source code: github.com/skriegman/reconfigurable_organisms/blob/master/networks.py

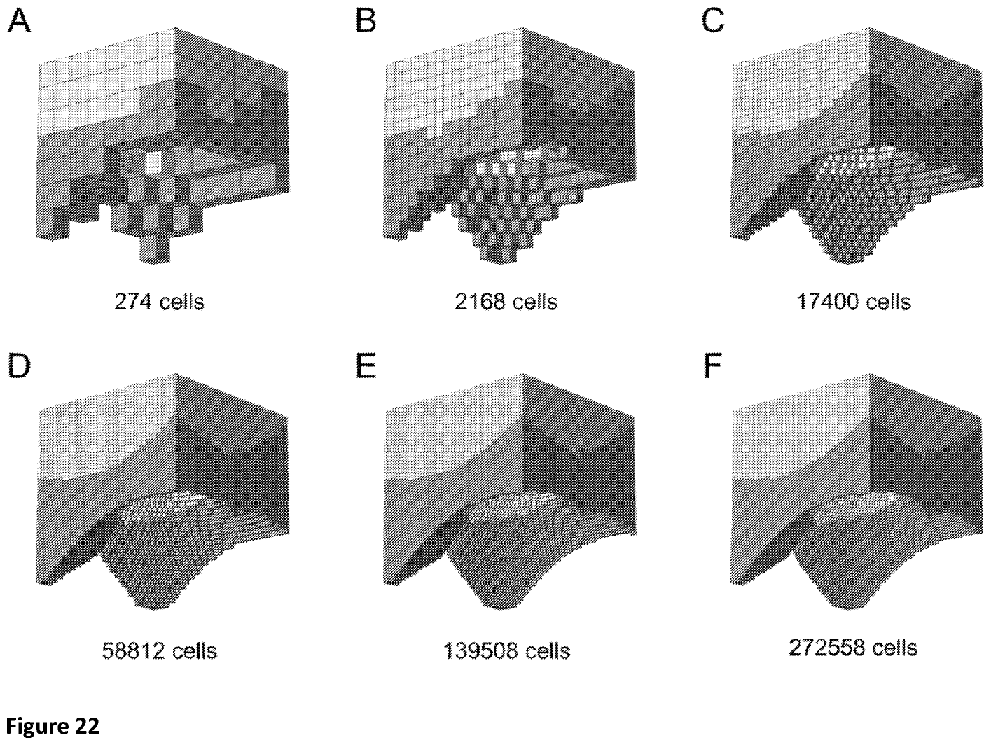

4.1. Genotype networks. Each configuration is genetically encoded using a Compositional Pattern-Producing Network, or CPPN (37), that maps the spatial coordinates of a 3D cartesian lattice to a single value indicating whether there is material at that location, and, if so, whether it is a contractile or passive cell. The genotype encoding is thus scale-free: A given genotype network can be mapped to arbitrary resolution coordinate space (FIGS. 2H and 22).

We chose this particular encoding because it tends to generate spatial regularities in structure, which are known to facilitate locomotion (38). In short, CPPNs are a special class of networks that use various activation (or interaction) functions that output regular patterns, such as sine waves and parabolas (instead of using only a single sigmoid or ramp function). CPPNs were originally proposed (37) as an abstraction of gene expression and embryonic development (rather than of neurons and brains), and we employ them as such here.

Input/output. The coordinates of each voxel are specified by their cartesian coordinates (x,y,z) and radial distances from the center of the lattice workspace (here, 8-by-8-by-7). These four coordinates (in addition to a bias term set equal to 1) are taken as input to the network. The inputs connect to (interact with) various regulatory and structural genes (vertices) by weighted edges (with real-valued scalar weights in the range −1 to +1) that multiply the input by the corresponding weight. Regulatory and structural genes sum all incoming weighted edges as input for their interaction function, which is here taken to be any of the following: sin( ), abs( ), square( ), sqrt(abs( )); and the negations of those four. The output expressions of regulatory genes are reweighted and passed onward to interact with additional regulatory genes (hidden nodes) or else with one of two structural genes (output nodes), whose output is thresholded at zero to determine (express) material presence and type, respectively. In some experiments (FIGS. 6 and 18), an additional, independent CPPN was used to determine the phase-offset (of open-loop volumetric actuation) at each voxel.

Mutation. Both the architectures and weights of these networks are evolved: mutations add, modify or remove a single randomly-selected vertex or edge. Offspring are created by performing six kinds of mutations (add vertex/edge, remove vertex/edge, modify vertex/edge), each of which is applied with probability 1/6. If none are selected (which occurs with probability (5/6) 6=0.33), one of the six is randomly chosen and applied. If a mutation is neutral (i.e., the resulting phenotypic structure is unchanged), another mutation is applied; after 1500 unsuccessful attempts at a non-neutral mutation, the 1500 neutral mutations are accepted. After mutation, the network is pruned of any erroneous edges and vertices that are not connected to the main graph (details about the evolutionary algorithm can be found in § 5.1).

Initialization, hyperparameters. Networks are initialized by fully connecting the input layer to the output, and normalizing the inputs (the coordinates of the workspace) to be between 0 and 1. Then, ten random vertices are added. Next, ten randomly-selected pairs of unconnected vertices are attached by a new edge, the weight of which is drawn from a uniform distribution between −1 and 1. (If the new edge creates a cyclic graph, erase it and retry, 999 times. If 1000 failed attempts occur, terminate.) After these additions, five randomly selected edges are removed. Then, 100 edges are randomly selected (with replacement) and their weights are mutated (serially) by adding a value drawn from a normal distribution with mean zero and standard deviation, s=0.5, clipping the new weight to be between −1 and 1. (Neutral mutations are permitted during this initialization process.) Finally, 100 vertices are likewise selected with replacement, and their interaction functions are replaced by functions randomly chosen from the set {±sin( ), ±abs( ), ±square( ), ±sqrt(abs( ))}. Hyperparameters were adopted from (24).

4.2. Morphological complexity. In this work, we have demonstrated the evolution of a CPPN encoding toward various geometries (FIG. 7) and topologies (FIG. 17) capable of open-loop locomotion and object manipulation in a simulated environment. However, these shapes tend to be relatively compact and simple, lacking more complex structures, such as branching limbs.

Previous work linked morphological complexity resulting from CPPN-genotypes with environmental complexity (39). We likewise observed the optimization of environment-specialized morphologies, such as the evolution of rudimentary end-effectors that hold an external object in place during its manipulation (FIG. 16).

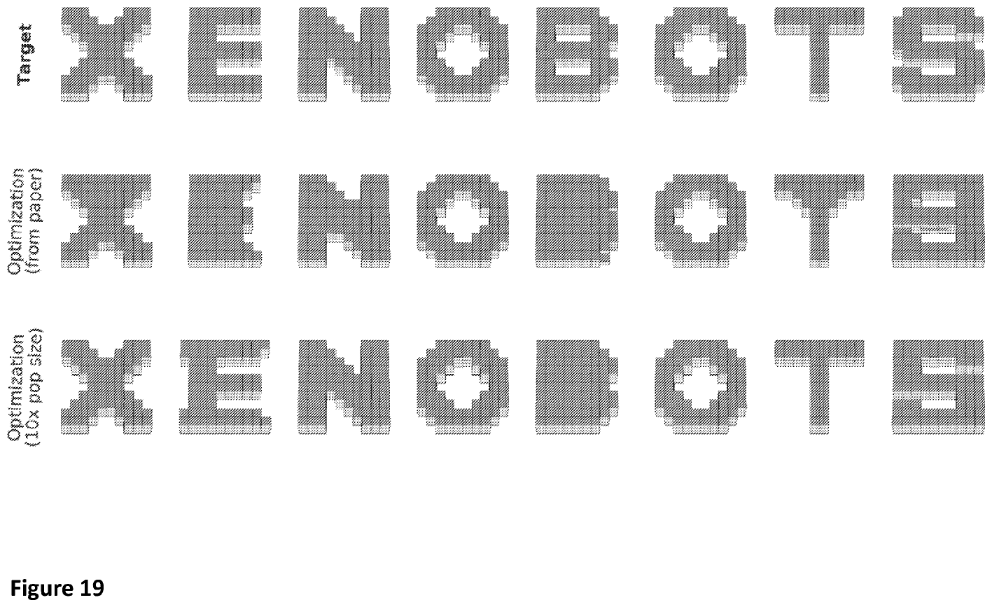

To demonstrate the flexibility of the encoding to achieve more complex morphologies as dictated by the task environment, we optimized CPPNs for a single evolutionary run to generate passive structures that match target shapes (FIG. 19). To do so, we used the same evolutionary algorithm and hyperparameters (detailed below in § 5) with a modified objective function: Performance was defined as the Hamming distance between binary matrices of the target and CPPN-output shapes (instead of locomotion velocity). In a separate experiment in which designs were challenged to throw an object (§ 10.3), we successfully evolved functional limbs by including an objective to maximize the design's surface-area to volume ratio, in addition to an objective for thrown object distance (FIG. 18). It would be of interest to explore, in future work, the genetic encodings and behavioral selections pressures that indirectly select for complex organism geometries.

5. Evolutionary Design.

This subsection outlines the evolutionary algorithm and its objective functions, analyzes the algorithm in smaller solution spaces, and compares evolution to a gradient-based approach.

5.1. The algorithm. A standard evolutionary algorithm was employed: Age-Fitness-Pareto Optimization, or AFPO (19). AFPO uses the concept of Pareto dominance and an objective of “age” (in addition to performance) intended to promote diversity among candidate designs and prevent premature convergence. “Age” is a clade-level attribute that counts the number of generations a clade has existed in the population: Each design can trace its ancestral roots back to a randomly configured, parentless individual, which was injected into the population, with age zero, at some previous generation. Thus the age roughly corresponds to the amount of search time spent in a particular area of design space.

One hundred independent evolutionary trials were conducted, each with a unique random seed (which is here set equal to the run number, 1-100), culminating in a unique run champion: the most performant design found, according to the goal function (FIGS. 6 and 7).

Each trial is initialized with a population of 50 randomly-configured designs with age zero. Every generation, the population is first doubled by creating modified copies of each individual in the population (offspring have the same age as their parent). The age of each individual is then incremented by one. Next, an additional randomly-configured individual (with age zero) is injected into the population (which now consists of 101 designs). Finally, selection reduces the population down to its original size (50 designs) according to the two objectives of performance (maximized) and age (minimized). That is, starting with the youngest and most performant designs, which are by definition nondominated, successive Pareto fronts are kept in their entirety until doing so would overfill the population past its original size (50 designs), at which point designs are added stochastically with probability proportional to their performance. This process of random variation and directed selection is repeated for 1000 generations.

A third objective was used in addition to performance and age: number of contractile voxels (minimized). This additional objective was added to ensure organisms had sufficient non-functional volumes that could be replaced by future non-actuating building blocks which may be required for specific tasks, such as wavelength perception (opsin-like detection), or metabolic pathways necessary for nutrient uptake and consumption. In spite of this objective, none of the run champions from the first pass for locomotion utilized passive voxels (FIG. 6).

Similarity to other architecture search algorithms. As mutations not only tune the parameters of an existing (parent) network, but can also add and remove genotype network structure (edges and vertices), the evolutionary algorithm is performing what is known as “architecture search” (40). Many evolutionary approaches to architecture search exist, such as NEAT (NeuroEvolution of Augmenting Topologies; (41)). However, NEAT evolves artificial neural networks, whereas we are evolving bodyplans. More specifically, we are evolving CPPNs (genotype; § 4.1) that encode bodyplans (phenotype). HyperNEAT (Hybercube-based NeuroEvolution of Augmenting Topologies; (42)) is an extension of the NEAT method which evolves CPPNs that typically, as the name suggests, encode neural network connectivity patterns. However, HyperNEAT has also been used to evolve CPPNs that encode soft robot bodyplans (43). We chose to evolve CPPNs using AFPO instead of HyperNEAT because the former is a much simpler algorithm than the latter. Despite the fact that it is more complex, it would be interesting to apply HyperNEAT to designing reconfigurable organisms; it could be that the additional machinery yields more performant designs. Source code: github.com/skriegman/reconfigurable_organisms/blob/master/tools/algorithms.py

5.2. Measuring performance. In all experiments, designs are allowed to settle under gravity for one second before the evaluation period begins. Just before the evaluation period starts, the initial center of mass of the design is recorded as (x₀, y₀, z₀).

For locomotion, the performance score was net displacement of the design's center of mass, in terms of euclidean distance: the square root of (x−x₀)²+(y−y₀)², where (x, y) is the final position of the design on the ground plane at the end of an evaluation period of 10 seconds. For object manipulation (§ 10.1), transport (§ 10.2), and expulsion (§ 10.3), the object's net displacement was tracked instead of the design's.

It is important to note that the formulation of the performance objective function must in some cases be refined in order to realize desired behavior. For example, an early version of the objective function for object transport (FIG. 17) intended to reward how far an object could be carried. However, because the lightweight object never touched (i.e., penetrated) the simulated ground plane, the optimizer discovered designs that dragged the object along the ground plane. This was corrected by constraining the object to be completely surrounded by tissue (§ 10.2).

5.3. Runtime. Each evolutionary trial optimized a population of 51 designs on a dual-processor, 12-core Intel E5-2650 v4 (i.e., 24 threads). No trial took less than 18 wall-clock hours (432 CPU hours) or more than 22 wall-clock hours (528 CPU hours) to evaluate 1000 generations of evolutionary improvement. The runtime varies because the algorithm is stochastic: some designs have more voxels than others and thus require more CPU-time. Because evaluating designs in simulation is the computational bottleneck, the algorithm is readily parallelizable: doubling CPU threads halved the wall-clock time (10 hours when tested using two Intel E5-2650 v4s).

5.4. Algorithm analysis. It is difficult to know what the optimal design is for large search spaces. So, we investigated a search space in which it was possible for us, given our computational resources, to determine exactly what the optimal design is (for a given random actuation pattern). We started with a 2×2×2 workspace and identified the optimal design for five different, random actuation patterns (FIG. 21A). The evolutionary algorithm found the optimal design for all five actuation patterns in 9129 evaluations (179 generations). In the slightly larger, 3×2×2 workspace, the evolutionary algorithm found the optimal design for all five actuation patterns in 4284 evaluations (84 generations) (FIG. 21B). In a 3×3×2 workspace, the algorithm took much longer, requiring 113,628 evaluations (2228 generations) to find the optimal design for all five actuation patterns (FIG. 21C). We could not determine the optimal design at 3×3×3, so we terminated the determination of optimal designs at this point. Source code: github.com/skriegman/reconfigurable_organisms/blob/master/exp/Algorithm_Analysis_EA.py

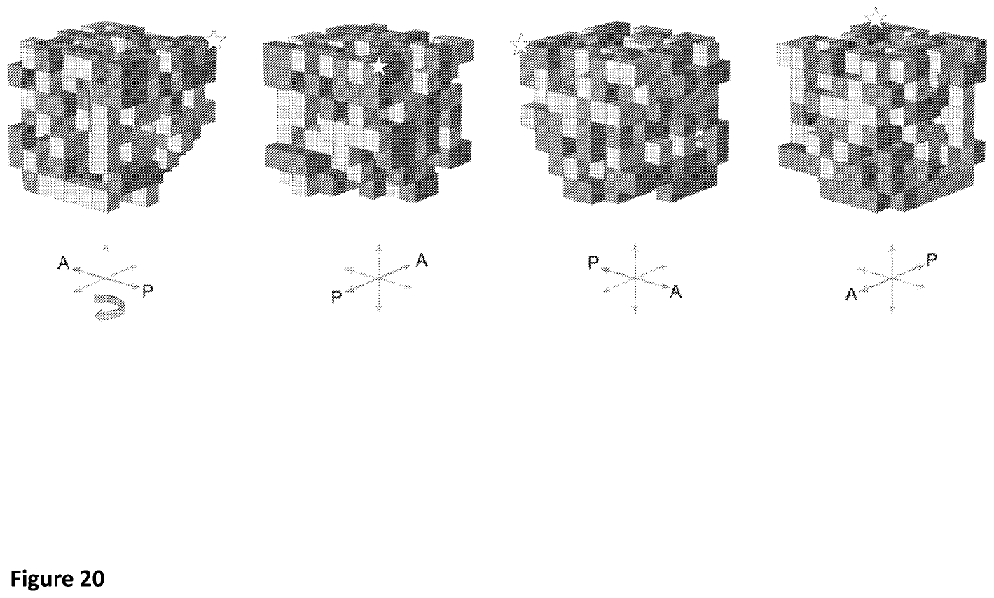

5.5. Comparison to a gradient-based approach. We employed an evolutionary algorithm because we have considerable experience using this particular algorithm to evolve soft robots in previous work. However, there are many forms of constrained optimization. One of the most common is stochastic gradient descent. So, we applied a stochastic gradient-based method (Parameter-exploring policy gradients; (44)) directly to the design problem. Typically the policy that is optimized is a vector of neural network weights (floating-point values). Here, the policy is a static arrangement of the discrete building blocks (ternary values). The algorithm samples the space of designs, evaluates their performance in silico, and estimates the gradient using a popular stochastic gradient descent optimizer (Adam; (45)). This algorithm yielded a less performant design (4.6 body lengths per minute; FIG. 20) that failed all three conditionals of the build filter (§ 7.2). Source code, which was adapted from (46), is available here: github.com/skriegman/reconfigurable_organisms/blob/master/exp/Algorithm_Analysis_SGD.p

The same population size as the evolutionary algorithm was used; all other hyperparameters were left at their default values as reported in (46). The policy vector of floating-point values was discretized to determine tissue type at each point in the workspace: values below −0.05 were left empty, values above 0.05 were encoded as passive voxels, and values in between −0.05 and 0.05 were encoded as contractile voxels. The two thresholds of −0.05 and 0.05 were set such that randomly-initialized designs at the beginning of optimization contained all three tissue types (the initial standard deviation is 0.10 by default).

This gradient-based algorithm quickly converges to suboptimal designs, even in very small search spaces (FIG. 21). To combat this premature convergence, we modified the algorithm to restart, every 1000 evaluations, from a different random initialization.

After 10,000 evaluations of search in the 2×2×2 workspace, the policy gradient algorithm found the optimal design for two of the five, random actuation patterns (40%). By restarting every 1000 evaluations, the modified algorithm was able to find the optimal design for four out of the five actuation patterns (80%) (FIG. 21A). In the 3×2×2 workspace, the algorithm did not find the optimal design for any of the five actuation patterns (0%). Modified to restart every 1000 evaluations, the algorithm found the optimal design for one of the five actuation patterns (20%) (FIG. 21B). In the 3×3×2 workspace, we allowed optimization to continue for much longer (140,000 evaluations), but the optimal design was not found for any of the five actuation patterns, with or without restarts (FIG. 21C). Source code: github.com/skriegman/reconfigurable_organisms/blob/master/exp/Algorithm_Analysis_SGDre.py

6. Updating Design Constraints.

This subsection describes how the design process is improved with feedback from the behavior of manufactured organisms.

On the first pass through the pipeline using the goal behavior of locomotion, the simulated environment consisted of an infinite plane and a gravitational acceleration of −9.81 m/s². Both the passive and contractile building blocks had a Young's modulus of 10′ Pa, a Poisson's Ratio of 0.35, and coefficients of static and dynamic friction of 1.0 and 0.5, respectively. Source code: github.com/skriegman/reconfigurable_organisms/blob/master/exp/Locomotion_pass1.py

The contractile voxels were volumetrically actuated (±50% rest volume) at 5 Hz. Although the cardiac tissue used to build reconfigurable organisms can only contract, simulated actuation also expanded voxels in volume because this produces more force, and thus faster locomoting designs in silico. We chose to call these voxels “contractile” to help clarify the match with contractile tissue. Future design-manufacture cycles of the pipeline could add a contraction-only actuation constraint in silico, however it was not necessary for successful transferral of behavior (§ 9.1).

The phase-offsets of actuation for each voxel (from a global, sinusoidal signal) were co-optimized with morphology, using two independent genotype networks (§ 4.1). Designs evolved propagating waves of volumetric actuation, yielding rapid locomotion via bounding gaits, with timeframes (on average, 47% of the gait cycle) in which no part of the design was in contact with the simulated ground plane (FIG. 6). However, when these designs were manufactured in vivo, they always kept part of their ventral surfaces in contact with the surface of the dish due to negative buoyancy.

This discrepancy was rectified by adding the following constraints to the simulated environment and actuation. On the second pass through the design pipeline, an aqueous environment was simulated by decreasing the gravitational acceleration to −0.1 m/s², and applying a drag force to surface voxels, assuming a fluid density of water (rho=1000 kg/m³) and a drag coefficient of C=1.5 for each exposed voxel face.

Voxels were simulated with half the Young's modulus (5×10⁶ Pa) and five times the length scale of those used during the first pass of the pipeline. This allowed a larger numerical integration timestep to be stable (see (22) for details), which greatly reduced the required CPU time of each evaluation.

Actuation frequency was reduced to 2 Hz to remove momentum effects (which are difficult to simulate accurately) and to better match the contraction rate of the cardiac tissue (˜1 Hz). The phase-offset of each voxel was randomized instead of optimized, which prevents designs from overfitting to a specific actuation policy. Each randomly-configured design injected into the population (with age zero; see § 5.1) was assigned 448 phase-offsets, randomly drawn from a uniform distribution between −π/2 and π/2, one value for every point (possible voxel location) in the 8×8×7 workspace. These phase-offsets were then hardcoded for the entire clade.

Evolutionary improvement within a clade thus occurred through changes in overall shape, and distribution of the passive and contractile voxels, to collectively derandomize the global movement produced by the random actuation. This reduced the dependence on precisely-timed actuation, which increased the likelihood of successful transferal from silicon to vivo. Source code: github.com/skriegman/reconfigurable_organisms/blob/master/exp/Locomotion_pass2.py

The surface contact behavior of designs generated on the second pass (FIG. 7) better matched that of the actual living systems: On the second pass through the design pipeline, run champions were, on average, in contact with the ground plane for 93.3% of their evaluation period, compared to just 52.7% on the first pass. The proportion of simulation time that designs are in contact with the ground was computed by recording (100 times per second) whether or not the z position of any voxels were penetrating the surface plane, using the following source code: github.com/skriegman/reconfigurable_organisms/blob/master/data_analysis/Time_in_contact_with_ground.py

7. Filtering Evolved Designs for Manufacture.

This subsection describes how designs were filtered based on their performance in silico, transfer potential, manufacturability under the current build method, and their scalability to more complex tasks in future deployments.

7.1. The robustness filter. The most performant designs (FIG. 7) were sorted by their robustness to random perturbations in their actuation (FIG. 8). Each design was then copied 20 times. The phase-offsets of the actuating voxels in each copy were independently mutated by adding a number that was drawn randomly (with random seeds 1-20) from a normal distribution with mean zero and standard deviation s=0.4π (which is 40% of the −π/2 to π/2 range of valid phase-offset values). This hyperparameter was selected to be large enough to scramble the original phase-offset value without being so large as to push all mutations up against the ±π/2 bounds. Designs that maintained the highest average performance across this actuation noise were passed, one by one, in order of their robustness ranking, to the build filter. Source code: github.com/skriegman/reconfigurable_organisms/blob/master/data analysis/Robustness_Filter.py

7.2. The build filter. The most robust designs were evaluated by their manufacturability under the current build method, which layers contiguous tissue regions sequentially, one on top of the other, with each layer filling the x,y plane (FIG. 10; § 8.2). Thus, the first criterion a design must meet for fabrication is that it must contain contiguous tissue regions that fill the dorsal and ventralmost x,y planes with just one of the two tissue types (i.e., no mixing within the dorsal and ventral layers). The design can be rotated in 3D space to satisfy this criterion (e.g., without mixing within the anterior and posteriormost layers).

Secondly, designs cannot contain arbitrarily small gaps in their geometry, because they are made of differentiating cells which will adhere to neighbors if they come into contact with each other. The minimal concavity was examined by producing organisms with progressively smaller shape deformations, then determining which persist across the lifespan of the organism, and which close due to tissue adherence/contraction, leading to loss of concavity. Preliminary work determined that concavities with a width of 100 μm or greater produced stable long term deformations suitable for biological building (FIG. 11). As the organisms typically have body diameters in the range 750±100 μm at the time of initial cutting and 850±100 μm after four days of healing, the minimal concavity width is 12%-14% of total body length.

Because larger designs can travel farther in the same amount of time as smaller ones, the in silico designs tend to fill at least one horizontal dimension of the 8×8×7 workspace (FIG. 7), resulting in a maximum length of 8 voxels. This equates to a minimal concavity width of a single voxel (1/8=12.5%). Thus, the second criterion for fabrication is that the design cannot contain gaps less than two voxels wide.

Finally, more complex tasks can require room for payloads (FIG. 17; § 10.2) or, in future, the addition of specialized cells for purposes other than locomotion, including sensory input, metabolism, memory, biosensors, etc. Also, contractile tissue incurs a high metabolic cost (compared to non-muscle tissue), which decreases the total lifetime of transferred designs. Thus, the third criterion for fabrication is that the design must be mostly (more than 50%) passive. Source code:github.com/skriegman/reconfigurable_organisms/blob/master/data_analysis/Build_Filter.py

8. Manufacturing Reconfigurable Organisms.

This subsection summarizes the current build method.

8.1. Cilia-driven organisms. During preliminary experiments aimed at testing the feasibility of in vivo designs, the manufactured organisms lacked contractile tissue and were instead propelled by cilia present on the surface of the body. These cilia-propelled spheres were manufactured in exactly the same way as the cardiomyocyte-driven organisms used to measure transfer success (§ 8.2), except the Notch intracellular domain (Notch-ICD) was not overexpressed through synthetic mRNA microinjection (this step inhibits multiciliated cell formation) prior to building. However, all of the ciliated organisms moved when released into the aqueous environment (albeit in unpredictable directions and at unpredictable speeds). Thus, measuring transferability between in vivo and in silico designs could produce false positives. In addition, the accurate modeling of swimming and fluid dynamics proved to be challenging in simulation, so we altered our build method to produce contractile based movement.

8.2. Cardiomyocyte-driven organisms. Contractile organisms were generated using two separate approaches (detailed in Methods and Materials). In the first, presumptive cardiomyocyte and epidermal cells were extracted from embryos and dissociated in calcium free magnesium free medium. Dissociated cells were then transferred to a 1 mm depression and the tissues were layered according to the desired in silico design. After two days of further development, final shaping was performed using a microcautery device and surgical forceps. For the second build method, the presumptive cardiomyocyte and epidermal tissues were not dissociated to the individual cell level. Instead, layers of tissue were stacked on top of one another, with the cardiomyocyte layers in the center. Shaping occurred in exactly the same manner, using a combination of microcautery and surgical forceps.

Both the dissociation and tissue layering method were employed during the course of the study, however, we chose to focus on the latter for movement-based assays. While this method results in lower accuracy of tissue placement (as specific numbers of cells could not be layered with precision), it significantly reduces total build time, allowing the investigator to produce approximately ten times the amount of organisms per unit time compared to the dissociation method.

9. Measuring Transfer Success.

This subsection explains how the behavior and structure of manufactured organisms were compared to those of the in silico design.

9.1. Transferal of behavior. The design was evaluated 25 times, resulting in 25 movement trajectories. Each time, the evolved set of phase-offsets for the actuating voxels was different, resulting in slightly different behavior (FIG. 4). This was done by adding to each of the original phase-offsets, a value that was drawn randomly from a normal distribution with mean zero and standard deviation s=0.4π (which is 40% of the −π/2 to π/2 range of valid phase-offset values). This hyperparameter was selected to be large enough to scramble the original phase-offset value without being so large as to push all mutations up against the ±π/2 bounds.

The numerical integration step size was 0.0032 seconds. For each random actuation pattern, the design was allowed to settle under gravity for 312 timesteps (one second), and then evaluated for 18750 additional timesteps (60 seconds), resulting in a total simulation time of 61 seconds.

Six reconfigurable organisms were built which embodied this design. They were imaged live for 10 minutes in 0.75×MMR at 20° C. using a Nikon SMZ-1500 microscope equipped with both top and substage illumination. Videos were captured using a Sony IMX234 at a sample rate of 30 fps. Behavioral trajectories were extracted using Noldus Ethovision 14 software. Each trajectory was then smoothed using a one-dimensional gaussian filter with a kernel standard deviation of 30 seconds.

Statistical analysis. In measuring transfer success, we made three comparisons (controlling for false discovery rate; (47)):

1. movement heading in vivo relative to in silico;

2. net displacement in vivo upright relative to inverted; and

3. net displacement in silico upright relative to inverted.

For movement heading, the data consist of the dichotomous outcomes: either the designed organism moved in the predicted direction, or not. Although organisms could move in any direction (0 to 360° relative to the predicted heading) we discretize the space into four directions of possible movement (forward, backward, left, right), only one of which is considered a success (forward). We have six realizations of the design in vivo (six separate organisms) that were each reset to the origin four times. As resets to the origin are clearly not independent observations, n=6. That is, we have six independent Bernoulli trials with probability of success q. Because there are four directions of possible movement, the null hypothesis is that organisms move in the predicted direction with probability q=1/4. All instances moved in the predicted direction, though one organism (and its four resets) was more or less sessile (with small amounts of movement in the predicted direction). The most conservative use of the data is to consider only five of the six realizations to be successful, and thus p=4.6×10⁻³. Controlling for false discovery rate (47), the null hypothesis is rejected at the 0.01 level of significance.

For net displacement, the data consist of paired replicates: anatomically-upright (“pretreatment”) and anatomically-inverted (“posttreatment”) observations from the same individual design. We are concerned with a shift in location (i.e., the median of the distribution of net displacement) due to inverting the design (the application of the “treatment”). We use a distribution-free signed-rank test (Wilcoxon). The null hypothesis asserts that each of the distributions for the differences (posttreatment minus pretreatment observations) is symmetrically distributed about 0, corresponding to no shift in location due to the treatment (inverting the design). In silico, p=9.7×10⁻⁵. Controlling for false discovery rate, the null hypothesis is rejected at the 0.001 level of significance.

In vivo, four organisms were evaluated five times while upright, and five times inverted. However, the recording equipment failed during one of the upright runs (Trial 2) for three of the organisms. This trial was simply removed from consideration for these three (i.e., displacement while inverted in Trial 2 was discarded where corrupted). The two other organisms were evaluated five times upright, but only once while inverted since these organisms generated little to no displacement when inverted, and motion tracking resources were limited. The single evaluation while inverted was therefore used as the posttreatment observation for each of the six upright (pretreatment) observations. In vivo, p=2.6×10⁻⁵. Controlling for false discovery rate, the null hypothesis is rejected at the 0.0001 level of significance.

In fact, only one of the six organisms was observed to produce appreciable forward movement while inverted. This anomaly was likely due to the cardiac tissue being layered deeper (more dorsally) than the other designs, resulting in a small amount of deformation on the dorsal surface. Source code for reproducing the in silico behavioral trajectories: github.com/skriegman/reconfigurable_organisms/blob/master/data_analysis/Transferal_from_silico_to_vivo.py. Source code for the statistical analysis: github.com/skriegman/reconfigurable_organisms/blob/master/data_analysis/Statistical_Analysis.py

9.2. Transferal of structure. The structure of organisms was compared to that of the in silico design using 2D images and Hausdorff distance.

To quantify the structural error (FIG. 12), lineage labeled organisms were created by harvesting tdTomato expressing cardiac progenitor tissue from one set of donor embryos, and GFPIII expressing passive epidermis tissue from a second set of donor embryos. Donor tissue was dissociated and transferred to 1 mm concave wells, placing both cell types in proportions and locations matching the in silico design. Two days later, the organism was then sculpted to the desired shape, and imaged in multiple orientations using a FITC filter set to visualize epidermal tissue (green) and a TRITC filter set to visualize cardiac progenitor (red) tissue. Channels were then overlaid in ImageJ to create the final composite image for analysis. K-means clustering was used to classify each pixel as one of three tissue types: contractile, passive, or none.

The Hausdorff distance between in vivo and in silico pixels was calculated for passive tissue only, then again for contractile tissue. The closest matching pixel (i.e., with the same tissue type) was found in silico for all in vivo pixels, and the closest matching pixel was likewise found in vivo for all in silico pixels. The Hausdorff distance is the largest such discrepancy between vivo and silico tissue coordinates, in terms of euclidean distance in microns. A small Hausdorff distance indicates that for every pixel in vivo there is a pixel of the same tissue type nearby in silico, and vice versa. After measuring the Hausdorff distance for both tissue types, the larger of the two is taken to be the structural error. Formally, the Hausdorff distance for tissue type 1 is defined as:

H₁=max{sup_{s∈S}_{₁}inf_{v∈V}_{₁}d(s,v),sup_{v∈V}_{₁}inf_{s∈S}_{₁}d(s,v)},

where S₁ and V₁ are the sets of in silico and in vivo pixels that were classified as the first tissue type, and d(s, v) is the euclidean distance between pixels s and v. Similarly, the Hausdorff distance for tissue type 2 is defined as:

H₂=max{sup_{s∈S}_{₂}inf_{v∈V}_{₂}d(s,v),sup_{v∈V}_{₂}inf_{s∈S}_{₂}d(s,v)},

where S₂ and V₂ are the sets of in silico and in vivo pixels that were classified as the second tissue type. The structural error is then:

max(H₁,H₂).

Before these comparisons can be made, however, the in vivo and in silico images need to be on a relatively consistent coordinate frame. Thus, images were auto-cropped such that the edge of the design fills the frame. This is done by converting each image to grayscale, and thresholding at 10/255 to create a binary image, thus isolating the organism against the dark background. Contours are then automatically drawn on the image by traversing the boundaries of each transition between black and white to find closed loops (each of which is a contour). A bounding box is drawn around the largest contour (by area), and the rest of the image is trimmed off. Finally, the cropped images were resized (downsampled) to a constant resolution (50×50).

A grid search was then performed to find the 3D perspective with the lowest structural error. The design was lowered/raised in elevation angle in the z plane, and spun around by azimuth angle in the x,y plane, in increments of 10° in each dimension. Then the 3D plot was saved as a 2D image and rotated again in increments of 10°. Since the tissue regions are classified via unsupervised clustering (and were thus assigned arbitrary labels), we take the regions with the largest overlap to be of the same type. This introduces the possibility of similarly shaped but different tissue types aligning to achieve the lowest structural error for a given in vivo perspective, but this did not occur in our experiments.

However, the rotation with the lowest structural error did not always respect the organism's anteroposterior alignment (the design was sometimes “facing” the opposite direction). So we restricted rotations of the design to better match the range of perspectives captured across the four images of the organism: the grid search was constrained to elevation angles between −40 and 60°, azimuth angles between −120 and −60°, and rotations of the resulting 2D image between 0 and 30°.

The average structural error achieved across all four images was 323 microns (38% of the organism's largest diameter) (FIG. 12).

Note that this preliminary method does not account for distortions resulting from flattening a 3D object to a 2D image (e.g., the moon terminator illusion). Future work will aim to capture many images of the organism from different perspectives, and use them to virtually reconstruct a 3D model for direct structural comparison. Source code: github.com/skriegman/reconfigurable_organisms/blob/master/data_analysis/Structural_Error.py

10. Applications.

This subsection summarizes four additional goal behaviors in which organisms can interact with an external object or other organisms.

10.1. Object manipulation. Introducing a single object to the environment, and altering the goal function to track the external object, instead of the design, yielded object manipulation—block pushing—in silico (FIG. 16). The new behavioral goal input into the pipeline was to maximize displacement of a 2×2×2 voxel object during an evaluation period of 30 seconds. This extended evaluation time of 30 seconds (instead of 10 sec) prevented the strategy of simply falling onto the object and hitting it forward ahead of the (often immobile) design. All other constraints from the second pass for locomotion were left in place: passive and randomly-actuating building blocks were reconfigured within an 8×8×7 workspace, and evaluated in a floored aqueous environment. Sixteen independent evolutionary runs were performed. In some designs, a primitive end-effector—a notch in the corner of the body—evolved to hold and manipulate the object as it was pushed along the floor. Source code: github.com/skriegman/reconfigurable_organisms/blob/master/exp/Object_Manipulation.py

10.2. Object transport. Some designs evolved for locomotion were hollow and could thus, in theory, be exapted to internally store the 2×2×2 voxel object as cargo, rather than push it externally. This realization led to the new goal of maximizing the euclidean distance of a carried object. Using a slightly larger, 10×10×9 workspace, a mask was placed on the design requiring the morphology to house the 2×2×2 object within a 4×4×4 voxel pouch. This yielded evolved object transport in silico (FIG. 17). All other constraints were identical to the second pass for locomotion (the evaluation period was reverted back to 10 sec). Sixteen evolutionary runs were performed.

Some modifications of the objective function were required to realize object transport. An early version of the objective function did not use masking to force the object to be inside the design at the center of the workspace. The object was instead free to be positioned anywhere in the workspace. Designs were then rewarded by net object displacement, with the stipulation that the evaluation period would be terminated if and when the object was detected to penetrate the floor. This is a standard way to ensure that a simulated object is touching the ground. However, given the buoyancy of the aqueous environment, the lightweight object would often touch but not penetrate the ground. What evolved were mostly variants that pushed or dragged the object across the ground plane. Source code: github.com/skriegman/reconfigurable_organisms/blob/master/exp/Object_Transport.py

10.3. Object expulsion. A non-locomotion-based goal was also supplied to the pipeline: maximize thrown object distance. In other words, evolve a catapult (48). We dropped a 2×2×2 voxel object (the projectile) from one voxel length above the 8×8×7 workspace, but no designs evolved to catch and throw the object forward. So we reverted actuation to be finely tuned (rather than randomized) by co-evolving phase-offset alongside structure, as in the first pass for locomotion. But, the evolved designs were relatively compact and lacked limbs for proper throwing. So, we induced direct selection pressure for limbs by incorporating an additional objective that maximized surface-area to volume ratio. In three of five evolutionary trials, limbs evolved which the design employed to throw the object (FIG. 18). However, all of the designs capable of throwing objects, failed to pass through the build filter (conditional B1 of the flowchart in FIG. 8) because they were composed entirely of muscle. Additionally, these designs rely on finely-tuned actuation, which is unlikely to transfer from in silico to in vivo. Source code: github.com/skriegman/reconfigurable_organisms/blob/master/exp/Object_Expulsion.py

10.4. Collective behavior. Multiple designs can be placed in a single environment instead of a single one. This can be done by simply relaxing the constraint which takes only the largest connected component to be the design. Alternatively, individually evolved designs can be evaluated together at a later stage in the pipeline. The latter method was implemented: five of the fastest locomoting designs (FIG. 7) were placed in the same environment amongst a grid of particulate matter in the form of 2×2×1 voxel particles, yielding spontaneous collective behavior and particle aggregation in silico (FIG. 14). Source code: github.com/skriegman/reconfigurable_organisms/blob/master/exp/Collective_Behavior.py

Spontaneous collective behavior and aggregation of external particles also occurred in vivo when multiple (10 to 15) organisms were placed together at the center of a single petri dish containing carmine dye (Sigma-Aldrich C1022-5G) (FIG. 15). A stock solution of carmine dye was created at a concentration of 0.01 g per 10 ml 0.75×MMR and vortexed for 10 seconds. Individual working solutions were then created in 1% agarose coated polystyrene petri dishes by diluting the stock 1:10, again in 0.75×MMR, for a final concentration of 0.001 g per 10 ml. Dishes containing the working solution were housed under an imaging microscope and allowed to settle for four hours at 22° C., creating a layer of particulate dye on the surface of the dish.

As a control, organisms were withheld from the dish, and the dye did not self aggregate after 1 or 24 hours.

11. Scaling the Pipeline.

This subsection describes how future design-manufacture pipelines may improve to scale the complexity and competence of reconfigurable organisms.

In the work reported here, actuation in silico was constrained to be random (§ 6) because it is not yet understood how to model the dynamics of cells in novel configurations and environments. This proved sufficient for some open-loop tasks, but not for others which required finer control (§ 10.3). Before a general understanding of the relationship between cell signaling and behavior is realized, behavioral biases of cells, or correlations between cells in certain configurations could be captured and distilled down into new constraints. However, in order to accurately incorporate the constraints of more realistic dynamics, the resolution of the simulation might need to be improved in future experiment designs.

Designs were constrained to 8-by-8-by-7 resolution (448 voxels) but the cardiomyocyte-driven organisms contained ˜5,000 cells (FIG. 22). The resolution of the workspace could be increased, but the required CPU-time scales with O(n+s²), where n is the total number of voxels, and s is the number of voxels that lie on the surface of the body, which are used to detect and resolve collisions. Such computational bottlenecks can be circumvented by incorporating an additional generator-filter pair along the pipeline that draws individuals from an evolving population of low-resolution designs (and/or low-resolution task environments) to evaluate at higher-resolution. In effect, the low-resolution population would serve as a computationally-cheaper surrogate model for higher-resolution designs and environments. However, it is currently not known how to guarantee that behavior generated in low resolution design space can be preserved when mapped onto higher resolution spaces.

At the time of writing, a GPU-accelerated version of the physics engine was released (creativemachineslab.com/titan-library) which can purportedly simulate millions of voxels in near real time. This raises the possibility of simulating reconfigurable organisms at the cell level, and exporting the design directly to a 3D bioprinting/cell-printing device containing multiple dispensers (each loaded with a bio-ink of an individual cell type), which could in principle exactly recapitulate the design in vivo. However, much work remains to adapt existing technologies for this purpose.

### SUPPLEMENTARY REFERENCES

36. The Online Encyclopedia of Integer Sequences (nos. A000162 and A001931). oeis.org/A001931
37. K. O. Stanley, Compositional pattern producing networks: a novel abstraction of development. Genetic programming and evolvable machines 8, 131-162 (2007).
38. J. Clune, B. Beckmann, C. Ofria, R. Pennock, Evolving coordinated quadruped gaits with the HyperNEAT generative encoding. Proceedings of the IEEE Congress on Evolutionary Computation (2009).
39. J. Auerbach, J. Bongard, Environmental Influence on the Evolution of Morphological Complexity in Machines. PLoS Computational Biology, 10, e1003399 (2014).
40. T. Elsken, J. Metzen, F. Hutter, Neural Architecture Search: A Survey. Journal of Machine Learning Research, 20, 1-21 (2019).
41. K. Stanley, R. Miikkulainen, Evolving Neural Networks through Augmenting Topologies. Evolutionary Computation, 10, 99-127 (2002).
42. K. Stanley, D. D'Ambrosio, J. Gauci, A Hypercube-Based Encoding for Evolving Large-Scale Neural Networks. Artificial Life, 15, 185-212 (2009).
43. N. Cheney, R. MacCurdy, J. Clune, H. Lipson, Unshackling Evolution: Evolving Soft Robots with Multiple Materials and a Powerful Generative Encoding. Proceedings of the 15th Annual Conference on Genetic and Evolutionary Computation, 167-174 (2013).
44. F. Sehnke, C. Osendorfer, T. RiickstieB, A. Graves, J. Peters, J. Schmidhuber, Parameter-exploring policy gradients. Neural Networks, 23, 551-559 (2010).
45. D. Kingma, J. Ba, Adam: A method for stochastic optimization. arXiv preprint arXiv: 1412.6980 (2014).
46. D. Ha, Evolving Stable Strategies (2017). blog.otoro.net/2017/11/12/evolving-stable-strategies
47. Y. Benjamini, Y. Hochberg, Controlling the false discovery rate: a practical and powerful approach to multiple testing. Journal of the Royal Statistical Society: Series B (Methodological), 57, 289-300 (1995).
48. N. Chaumont, R. Egli, C. Adami, Evolving virtual creatures and catapults. Artificial life, 13, 139-157 (2007).

TABLE 1
Biological Reagents

| | | |
|---|---|---|
| 1 | Xenopus frogs and | Nasco LM00490, LM00531, |
| | embryos: | LM00456 |
| 2 | Standard Xenopus media | 1M NaCl, 20 mM KCl, 10 mM MgSO₄, |
| | (10x stock): | 20 mM CaCl₂ dihydrate, |
| | | 20 mM HEPES, ph 7.4. |
| 3 | 0.75x Xenobot media: | Diluted from 10x Stock Xenopus media |
| 4 | Cell dissociation media: | 50.3 mM NaCl, 0.7 mM KCl, 9.2 mM |
| | | Na₂HPO₄, 0.9 mM KH₂PO₄, |
| | | 2.4 mMNaHCO₃, 1.0 mM EDTA, |
| | | pH 7.3. |
| 5 | tdTomato lineage tracer: | Waldner C, Roose M. Ryffel G U. |
| | | 2009. BMC Dev Bio. 9(1): 37. |
| 6 | GFPIII lineage tracer: | Zernicka-Goetz M, et al. 1996. |
| | | Development. 1; 122(12): 3719-24. |
| 7 | NotchICD cilia | Deblandre G A, et al. 1999. |
| | inhibiting construct: | Development. 1; 126(21): 4715-28. |
| 8 | 1% agarose substrate for | VWR EM-2125 |
| | cap movement trials: | |
| 9 | Petri Dish, | Fisher FB0875713A |
| | 60 mm × 15 mm | |
| | polystyrene: | |
| 10 | Carmine dye: | Sigma-Aldrich C1022-5G |
| 11 | Gentamicin: | ThermoFisher Scientific, 15710072 |

TABLE 2
Shaping Tools

| | | |
|---|---|---|
| 1 | Microsurgery forceps: | Dumont, 11241-30 #4 |
| 2 | Forcep sharpening stone: | Fine Science Tools 29008-22 |
| 3 | Microcautery device: | Protech International Inc., |
| | | MC-2010 |
| 4 | 13 micron cautery electrode: | Protech International, 13-Y1 |
| 5 | Binocular dissecting | VWR 19000-852 |
| | microscope: | |

TABLE 3
Imaging

| | | |
|---|---|---|
| 1 | Fluorescent microscope: | Nikon SMZ-1500 |
| 2 | Microscope camera: | QImaging Retiga 2000R CCD |
| 3 | Ipod touch for time lapse | Apple Inc. |
| | imaging: | |
| 4 | Ipod microscope mount: | Gosky Binocular adapter |
| | | B013D2ULO6 |
| 5 | ImageJ image analysis | National Institutes of Health |
| | software: | |
| 6 | Motion tracking software: | Noldus Ethovision 14 |

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. An engineered multicellular organism comprising an aggregate of cells comprising passive cells and contractile cells, wherein the organism moves when the contractile cells are actuated.

2. The organism of claim 1, wherein the organism consists of biological material and/or does not comprise any inorganic material, for example as a scaffold.

3. The organism of claim 1, wherein the organism comprises a sensor for detecting a target molecule.

4. The organism of claim 1, wherein the cells of the organism self-assemble.

5. The organism of claim 1, wherein the organism moves when the contractile cells are actuated, optionally wherein the organism moves substantially linearly.

6. The organism of claim 1, wherein the organism moves linearly at a rate of at least about 1 mm/minute when the contractile cells are actuated.

7. The organism of claim 1, wherein the organism moves only when the organism is in an upright position and not when the organism is in an inverted position.

8. The organism of claim 1, wherein the passive cells and/or the contractile cells are obtained from pluripotent blastula cells.

9. The organism of claim 1, wherein the passive cells and/or the contractile cells are engineered to express a heterologous molecule.

10. The organism of claim 9, wherein the heterologous molecule is one or more of a therapeutic agent, an enzyme that metabolizes a target substrate, and receptor for a target ligand.

11-14. (canceled)

15. The organism of claim 1, wherein the organism is configured to have a cavity for capturing and/or transporting a target object, and/or wherein the organism comprises one or more protrusions that facilitate movement of the organism, optionally vertical protrusions that contact a surface and facilitate movement of the organism.

16. (canceled)

17. The organism of claim 1, wherein the organism comprises one or more of vertebrate cells, invertebrate cells, and amphibian cells.

18-23. (canceled)

24. A method for delivering a therapeutic agent to a subject in need thereof, the method comprising engineering the organism of claim 1 to express the therapeutic agent and administering the organism to the subject.

25. A method for removing a target substrate from an environment, the method comprising engineering the organism of claim 1 to express an enzyme that metabolizes the target substrate and placing the organism in the environment.

26. A method for detecting a target ligand in a sample, the method comprising engineering the organism of claim 1 to express a receptor for the target ligand and place the organism in the sample, wherein the organism generates a signal after the receptor binds the target ligand.

27. A system for designing the organism of claim 1, the system comprising at least one hardware processor that is programmed to:
(i) generate a design space comprising a plurality of configurations of a polycube, the polycube comprising passive voxels, contractile voxels, and null voxels;
(ii) simulate movement or the lack thereof of the configurations based on actuation of the contractile voxels of the configurations;
(iii) generate a report reporting movement or lack thereof for the configurations;
and optionally
(iv) selectively delete configurations that have movement capabilities less than the average movement capabilities of a current set of configurations, selectively copy and randomly modify the remaining configurations, and repeat this process to effect evolution of the set of configurations.

28. The system of claim 27, wherein movement is simulated utilizing a physics engine that: (a) models interactions between adjacent voxels and interactions between voxels and a ground surface plane; (b) simulates volumetric actuation resulting from contractile cells; and (c) detects and resolves collisions between voxels.

29. The system of claim 27, wherein movement is simulated by (a) generating an input for the simulation by encoding each configuration as a network that maps the spatial coordinates of a 3D cartesian lattice of the configuration to a single value indicating whether there is a voxel associated with that value (i.e., the location is not a null voxel) and whether there is a contractile voxel or passive voxel associated with that value, wherein the input comprises each voxel's coordinate values, and each voxel's coordinate values comprise each voxel's cartesian coordinates and each voxel's radial distance from the center of the 3D cartesian lattice; (b) generating an output for the simulation by applying a function to each voxel's coordinate values based on volumetric actuation resulting from contractile cells.

30-34. (canceled)

35. A method for designing the organism of claim 1, the method comprising:
(i) generating a design space comprising a plurality of configurations of a polycube, the polycube comprising passive voxels, contractile voxels, and null voxels;
(ii) simulating movement or the lack thereof of the configurations based on actuation of the contractile voxels of the configurations;
(iii) generating a report reporting movement or lack thereof for the configuration; and optionally
(iv) selectively delete configurations that have movement capabilities less than the average movement capabilities of a current set of configurations, selectively copy and randomly modify the remaining configurations, and repeat this process to effect evolution of the set of configurations.

36. The method of claim 35, wherein movement is simulated utilizing a physics engine that: (a) models interactions between adjacent voxels and interactions between voxels and a ground surface plane; (b) simulates volumetric actuation resulting from contractile cells; and (c) detects and resolves collisions between voxels.

37-42. (canceled)
